SEQUENCE LISTING (51 sequences)

SEQ ID NO: 1 - DNA, 3006 bp, Homo sapiens
  source 1..3006: mol_type = genomic DNA
cctggttttctgtctgaatcaactgtacgagttaaacagttttatttcgaaattaaccaacataaacaacaacaacaacaacaacattcactacccattccctcgttaggttggaaaaccagtctgaaaaaataaatacaatctgtgaccatgaaaaggagcaatcacttatttttcccatatgttttgaacattatttttaaaaatagattgggaatctggagggtaaaatgccgggtcccttcccgagcccttagagcccgaacgttgtcgaatcggtcaatgtccaccccgctgctcacctctgtcccagcagcgccggggccagcgcgccctcccgccgcgtctgcggagctgcgggaaaagcaggtccccggggggtatcgagtgtccagggatatccacgcagacatccttgtacttgcaaatacaaagaaacacacaggacagagatcaggctcgcgccagggctcgatctcctcggggctcagggttggaggccgtacacagcctggacacaaactgagccaaagagtttatttttagcaaataagatatgagtttttagaaaattgcttcgaatagcaaatccgagaaacattatttcaaaacgttggaatcaaaggaaatcaaatgcccacaacccatttccgtcagtgcttacaaatgagcttctgcaaaacccagtccacttaaacatttggtcggaactcacgaaaagaaccaaaagcaaggcattccgagtcctggcgggggttgagcggccgggcggacgcctggctatctcttctgggagtcgcgcattcgatcgcattcaggttaaaatttaaagcccctggatcctgctccagtcgccagcctctccattccagagttttctaccttcagccttttccaaatgcggccacgctgaaactgggagccgccgcagtgattgggggaggcaaagtggaacgtaataaaagtgactaaattggatgagaacggtttctggaccttctcggtagcaatttaaaattcagaagtgtttagaagcaatttacaacgaagaatgaaaaacgaagaattctcgccaacttcctgaattccttgccccctcccccgcactgccaggccctgtcccgctcccccaggagccacaagttcacaactcacagttacgtgcagaggaaacaacgcttagctacgaggtctcaccatctctggaaaccatactttacaaggcacctctccaccccagttcaactctgcacaggctcctcttccttccctaactgggaaaacacgaaaatagaaacaaaacaaaaaaccagagcctaaacgctttctaggagaccgaccttcagaaaatcatttacttggatttacctgggcggggggtagggggtggggtcggggtaaagtataaaaattcattctcacaaaaacaacgcctcttgtcctaaagtctccctcggcagccttctggggtcacgtctgtcggaagaaatcctttctgtaagcgaaagggagcgagacatatatcacggctcctgcagcggacgagcctgcggccgcgcgagttgccgctgacagatcggcgctgataaccccgcgacgttcatttccagtcccttttgcccgctagggaccgtatcagtttgtacagagttgtgataactgtttggagggagcgaagagggggtgtgtatatgaacgtgggggtggggaagcagcatttagcgctgggtccagggaaggtgacaccccctcttcgcagtgacttatctgtgacttacctgaaactctccagggaaatatcagaaactctccagggacatcaaaagttggagagcgtcctcggacacgactgatgtggaagcccttttccattctgcgtaccccatagactacctttccgtacatgacgacccgagttaaagttcccaaaggtcagctggggaatgttgtcttgaggttcccgaaaccaccacgacctgagccgtagcatcccgagatagggtcggagaaagtttaaggtcggtctcacacaacttcagggcaaaaagcgcatttgctgtgaaggggctaggcgggggttggggggcgggggggcgcagagcacgttcccccttccttctgggggcgtgctgacccaaggtctggagcgcccctcccgccgcggcggcggcgcgggactgtgcactgccaactcctcccgtgccaaggctccctccccctccctcgtgtgggtgtgtgtgtgcgactgcgggagcgggagggtgcaccgccgctggatgggtgcgggtcgctagccaggtcaggcgttctgtggcctccgcccctcccccggggtccctgggctcggtgccccccgggtggactcgcccccactccggggacagggcttgcggctcagcccacgcccgcaggcagcgcggctctcattgtctgcctgggcggcgcgctcccctcccccctcgccgtcccctccccaccctcttttctctcctcccctcgatccctcctcctcctcttcacctccagcgcccagctgctcgctgagcgcagttccgacccacagcctggcacccttcggcgagcgctgtttgtttagggctcggtgagtccaatcaggagcccaggctgcagttttccggcagagcagtaagaggcgcctcctctctcctttttattcaccagcagcgcggcgcagaccccggactcgcgctcgcccgctggcgccctcggcttctctccgcgcctgggagcaccctccgccgcggccgttctccatgcgcagcgcccgcccgaggttcggctgcttcctcctcgcgcgatctccccgctttcctcccctccacccctactcgtcgcgggcccctggcctccggctctccggccctctccaggccgcggtcggctccgacggtgtttccctccttccctccgggcgggctgaggcggctcccaggctcccgggacaccgcggaccaacttctagtctctttttctctcctcgaagcg

SEQ ID NO: 2 - RNA, 980 nt, synthetic construct
  source 1..980: mol_type = other RNA
aaacagttatcacaactctgtacaaactgatacggtccctagcgggcaaaagggactggaaatgaacgtcgcggggttatcagcgccgatctgtcagcggcaactcgcgcggccgcaggctcgtccgctgcaggagccgtgatatatgtctcgctccctttcgcttacagaaaggatttcttccgacagacgtgaccccagaaggctgccgagggagactttaggacaagaggcgttgtttttttagggaaggaagaggagcctgtgcagagttgaactggggtggagaggtgccttgtaaagtatggtttccagagatggtgagacctcgtagctaagcgttgtttcctctgcacgtaacttttcagcgtggccgcatttggaaaaggctgaaggtagaaaactctggaatggagaggctggcgactggagcaggatccaggggctttaaattttaacctgaatgcgatcgaatgcgcgactcccagaagagatagccaggcgtccgcccggccgctcaacccccgccaggactcggaatgccttgcttttggttcttttctacaaggatgtctgcgtggatatccctggacactcgataccccccggggacctgcttttcccgcagctccgcagacgcggcgggagggcgcgctggccccggcgctgctgggacagaggtgagcagcggggtggacattgaccgattcgacaacgttcgggctctaagggctcgggaagggacccggcattttaccctccagattcccaatctatttttaaaaataatgttcaaaacatatgggaaaaataagtgattgctccttttcatggtcacagattgtatttattttttcagactggttttccaacctaacgagggaatgggtagtgaatgttgttgttgttgttgttgtttatgttggttaatttcgaaataaaactgtttaactcgtacagttgattcagacagaaaacca

SEQ ID NO: 3 - RNA, 1217 nt, synthetic construct
  source 1..1217: mol_type = other RNA
aaacagttatcacaactctgtacaaactgatacggtccctagcgggcaaaagggactggaaatgaacgtcgcggggttatcagcgccgatctgtcagcggcaactcgcgcggccgcaggctcgtccgctgcaggagccgtgatatatgtctcgctccctttcgcttacagaaaggatttcttccgacagacgtgaccccagaaggctgccgagggagactttaggacaagaggcgttgtttttttagggaaggaagaggagcctgtgcagagttgaactggggtggagaggtgccttgtaaagtatggtttccagagatggtgagacctcgtagctaagcgttgtttcctctgcacgtaactgtgagttgtgaacttgtggctcctgggggagcgggacagggcctggcagtgcgggggagggggcaaggaattcaggaagttggcgagaattcttcgtttttcattcttcgttgtaaattgcttctaaacacttctgaattttaaattgctaccgagaaggtccagaaaccgttctcatccaatttagtcacttttattacgttccactttgcctcccccaatcactgcggcggctcccagtttcagcgtggccgcatttggaaaaggctgaaggtagaaaactctggaatggagaggctggcgactggagcaggatccaggggctttaaattttaacctgaatgcgatcgaatgcgcgactcccagaagagatagccaggcgtccgcccggccgctcaacccccgccaggactcggaatgccttgcttttggttcttttctacaaggatgtctgcgtggatatccctggacactcgataccccccggggacctgcttttcccgcagctccgcagacgcggcgggagggcgcgctggccccggcgctgctgggacagaggtgagcagcggggtggacattgaccgattcgacaacgttcgggctctaagggctcgggaagggacccggcattttaccctccagattcccaatctatttttaaaaataatgttcaaaacatatgggaaaaataagtgattgctccttttcatggtcacagattgtatttattttttcagactggttttccaacctaacgagggaatgggtagtgaatgttgttgttgttgttgttgtttatgttggttaatttcgaaataaaactgtttaactcgtacagttgattcagacagaaaa

SEQ ID NO: 4 - RNA, 1787 nt, synthetic construct
  source 1..1787: mol_type = other RNA
aacccccgcctagccccttcacagcaaatgcgctttttgccctgaagttgtgtgagaccgaccttaaactttctccgaccctatctcgggatgctacggctcaggtcgtggtggtttcgggaacctcaagacaacattccccagctgacctttgggaactttaactcgggtcgtcatgtacggaaaggtagtctatggggtacgcagaatggaaaagggcttccacatcagtcgtgtccgaggacgctctccaacttttgatgtccctggagagtttctgatatttccctggagagtttcagaaaggatttcttccgacagacgtgaccccagaaggctgccgagggagactttaggacaagaggcgttgtttttgtgagaatgaatttttatactttaccccgaccccacccctaccccccgcccaggtaaatccaagtaaatgattttctgaaggtcggtctcctagaaagcgtttaggctctggttttttgttttgtttctattttcgtgttttcccagttagggaaggaagaggagcctgtgcagagttgaactggggtggagaggtgccttgtaaagtatggtttccagagatggtgagacctcgtagctaagcgttgtttcctctgcacgtaactgtgagttgtgaacttgtggctcctgggggagcgggacagggcctggcagtgcgggggagggggcaaggaattcaggaagttggcgagaattcttcgtttttcattcttcgttgtaaattgcttctaaacacttctgaattttaaattgctaccgagaaggtccagaaaccgttctcatccaatttagtcacttttattacgttccactttgcctcccccaatcactgcggcggctcccagtttcagcgtggccgcatttggaaaaggctgaaggtagaaaactctggaatggagaggctggcgactggagcaggatccaggggctttaaattttaacctgaatgcgatcgaatgcgcgactcccagaagagatagccaggcgtccgcccggccgctcaacccccgccaggactcggaatgccttgcttttggttcttttcgtgagttccgaccaaatgtttaagtggactgggttttgcagaagctcatttgtaagcactgacggaaatgggttgtgggcatttgatttcctttgattccaacgttttgaaataatgtttctcggatttgctattcgaagcaattttctaaaaactcatatcttatttgctaaaaataaactctttggctcagtttgtgtccaggctgtgtacggcctccaaccctgagccccgaggagatcgagccctggcgcgagcctgatctctgtcctgtgtgtttctttgtatttgcaagtacaaggatgtctgcgtggatatccctggacactcgataccccccggggacctgcttttcccgcagctccgcagacgcggcgggagggcgcgctggccccggcgctgctgggacagaggtgagcagcggggtggacattgaccgattcgacaacgttcgggctctaagggctcgggaagggacccggcattttaccctccagattcccaatctatttttaaaaataatgttcaaaacatatgggaaaaataagtgattgctccttttcatggtcacagattgtatttattttttcagactggttttccaacctaacgagggaatgggtagtgaatgttgttgttgttgttgttgtttatgttggttaatttcgaaataaaactgtttaactcgtacagttgattcagacag

SEQ ID NO: 5 - RNA, 795 nt, synthetic construct
  source 1..795: mol_type = other RNA
aaagagactagaagttggtccgcggtgtcccgggagcctgggagccgcctcagcccgcccggagggaaggagggaaacaccgtcggagccgaccgcggcctggagagggccggagagccggaggccaggggcccgcgacgagtaggggtggaggggaggaaagcggggagatcgcgcgaggaggaagcagccgaacctcgggcgggcgctgcgcatggagaacggccgcggcggagggtgctcccaggcgcggagagaagccgagggcgccagcgggcgagcgcgagtccggggtctgcgccgcgctgctggtgaataaaaaggagagaggaggcgcctcttactgctctgccggaaaactgcagcctgggctcctgattggactcaccgagccctaaacaaacagcgctcgccgaagggtgccaggctgtgggtcggaactgcgctcagcgagcagctgggcgctggagttgtgtgagaccgaccttaaactttctccgaccctatctcgggatgctacggctcaggtcgtggtggtttcgggaacctcaagacaacattccccagctgacctttgggaactttaactcgggtcgtcatgtacggaaaggtagtctatggggtacgcagaatggaaaagggcttccacatcagtcgtgtccgaggacgctctccaacttttgatgtccctggagagtttctgatatttccctggagagtttcagtttcagcgtggccgcatttggaaaaggctgaaggtagaaaactctggaatggagaggctggcgactggag

SEQ ID NO: 6 - RNA, 710 nt, synthetic construct
  source 1..710: mol_type = other RNA
aaaggctgaaggtagaaaactctggaatggagaggctggcgactggagcaggatccaggggctttaaattttaacctgaatgcgatcgaatgcgcgactcccagaagagatagccaggcgtccgcccggccgctcaacccccgccaggactcggaatgccttgcttttggttcttttccctgttatctaggccacccaatgaccgacgaaagattttccctccctgcttctcccgtggccgctttaaaccaaataaatttagctattaagggccaagatgtctgacaaaagatcgaaaggtgtctttccgtgatcgcttaaagaagcgaaggagaagcttgcggctctcaaatcccgataaagaaattccgcgccggcgccggcctccctccccgcggcctggttgctccctgacgccctgagaccccgacttcggcggccgcagacctcggacgggcccggcgggacctgtggctgccgcctggggaaggtggaggtccccgggctgcggggccagggagtgccaggccctaggagacgggcggacaagggggtccccgggagctctggagctccaggtgggagctgggtatcctaaaacggcccccgtgcacttcgcggaaaaactaagccaagctcagggggccgaaattaccactccgttttgattattttctgatactaaataataaagataaaaccaaactgcc

SEQ ID NO: 7 - RNA, 419 nt, synthetic construct
  source 1..419: mol_type = other RNA
acgtcaggtggtggcggcggaaacttgcaggtcgcccccacccccagcgccggctcccgccggcggccacttcggggaaggaccgcgcggagccagcgccggggtccccgggcggtcggaatcgaggcccggcgcgttgcgcaggccaggcccggcccggaggccccttgcccggcgcagcccccggccgacccccgtgcgccgccggcggccgccagcacggcgcggaggtgggctagcggcggcggcgcacgaaaggtcaccgcggtggcattcctctccgcgctgtttacgtgacttaatgtactcgctgcgccgcggcctgttatctaggccacccaatgaccgacgaaagattttccctccctgcttctcccgtggccgctttaaaccaaataaatttagctattaagggccaa

SEQ ID NO: 8 - RNA, 1659 nt, synthetic construct
  source 1..1659: mol_type = other RNA
agttatcacaactctgtacaaactgatacggtccctagcgggcaaaagggactggaaatgaacgtcgcggggttatcagcgccgatctgtcagcggcaactcgcgcggccgcaggctcgtccgctgcaggagccgtgatatatgtctcgctccctttcgcttacagaaaggatttcttccgacagacgtgaccccagaaggctgccgagggagactttaggacaagaggcgttgtttttgtgagaatgaatttttatactttaccccgaccccaccccctaccccccgcccaggtaaatccaagtaaatgattttctgaaggtcggtctcctagaaagcgtttaggctctggttttttgttttgtttctattttcgtgttttcccagttagggaaggaagaggagcctgtgcagagttgaactggggtggagaggtgccttgtaaagtatggtttccagagatggtgagacctcgtagctaagcgttgtttcctctgcacgtaactgtgagttgtgaacttgtggctcctgggggagcgggacagggcctggcagtgcgggggagggggcaaggaattcaggaagttggcgagaattcttcgtttttcattcttcgttgtaaattgcttctaaacacttctgaattttaaattgctaccgagaaggtccagaaaccgttctcatccaatttagtcacttttattacgttccactttgcctcccccaatcactgcggcggctcccagtttcagcgtggccgcatttggaaaaggctgaaggtagaaaactctggaatggagaggctggcgactggagcaggatccaggggctttaaattttaacctgaatgcgatcgaatgcgcgactcccagaagagatagccaggcgtccgcccggccgctcaacccccgccaggactcggaatgccttgcttttggttcttttcgtgagttccgaccaaatgtttaagtggactgggttttgcagaagctcatttgtaagcactgacggaaatgggttgtgggcatttgatttcctttgattccaacgttttgaaataatgtttctcggatttgctattcgaagcaattttctaaaaactcatatcttatttgctaaaaataaactctttggctcagtttgtgtccaggctgtgtacggcctccaaccctgagccccgaggagatcgagccctggcgcgagcctgatctctgtcctgtgtgtttctttgtatttgcaagtacaaggatgtctgcgtggatatccctggacactcgataccccccggggacctgcttttcccgcagctccgcagacgcggcgggagggcgcgctggccccggcgctgctgggacagaggtgagcagcggggtggacattgaccgattcgacaacgttcgggctctaagggctcgggaagggacccggcattttaccctccagattcccaatctatttttaaaaataatgttcaaaacatatgggaaaaataagtgattgctccttttcatggtcacagattgtatttattttttcagactggttttccaacctaacgagggaatgggtagtgaatgttgttgttgttgttgttgtttatgttggttaatttcgaaataaaactgtttaactcgtacagttgattcagacagaaaacca

SEQ ID NO: 9 - RNA, 848 nt, synthetic construct
  source 1..848: mol_type = other RNA
gatctgtcagcggcaactcgcgcggccgcaggctcgtccgctgcaggagccgtgatatatgtctcgctccctttcgcttacagaaaggatttcttccgacagacgtgaccccagaaggctgccgagggagactttaggacaagaggcgttgtttttttagggaaggaagaggagcctgtgcagagttgaactggggtggagaggtgccttgtaaagtatggtttccagagatgtttcagcgtggccgcatttggaaaaggctgaaggtagaaaactctggaatggagaggctggcgactggagcaggatccaggggctttaaattttaacctgaatgcgatcgaatgcgcgactcccagaagagatagccaggcgtccgcccggccgctcaacccccgccaggactcggaatgccttgcttttggttcttttctacaaggatgtctgcgtggatatccctggacactcgataccccccggggacctgcttttcccgcagctccgcagacgcggcgggagggcgcgctggccccggcgctgctgggacagaggtgagcagcggggtggacattgaccgattcgacaacgttcgggctctaagggctcgggaagggacccggcattttaccctccagattcccaatctatttttaaaaataatgttcaaaacatatgggaaaaataagtgattgctccttttcatggtcacagattgtatttattttttcagactggttttccaacctaacgagggaatgggtagtgaatgttgttgttgttgttgttgtttatgttggttaatttcgaaataaaactgtttaactcgtacagttgattcagacagaaaa

SEQ ID NO: 10 - RNA, 785 nt, synthetic construct
  source 1..785: mol_type = other RNA
agaaaggatttcttccgacagacgtgaccccagaaggctgccgagggagactttaggacaagaggcgttgtttttggaaggaagaggagcctgtgcagagttgaactggggtggagaggtgccttgtaaagtatggtttccagagatggtgagacctcgtagctaagcgttgtttcctctgcacgtaacttttcagcgtggccgcatttggaaaaggctgaaggtagaaaactctggaatggagaggctggcgactggagcaggatccaggggctttaaattttaacctgaatgcgatcgaatgcgcgactcccagaagagatagccaggcgtccgcccggccgctcaacccccgccaggactcggaatgccttgcttttggttcttttctacaaggatgtctgcgtggatatccctggacactcgataccccccggggacctgcttttcccgcagctccgcagacgcggcgggagggcgcgctggccccggcgctgctgggacagaggtgagcagcggggtggacattgaccgattcgacaacgttcgggctctaagggctcgggaagggacccggcattttaccctccagattcccaatctatttttaaaaataatgttcaaaacatatgggaaaaataagtgattgctccttttcatggtcacagattgtatttattttttcagactggttttccaacctaacgagggaatgggtagtgaatgttgttgttgttgttgttgtttatgttggttaatttcgaaataaaactgtttaactcgta

SEQ ID NO: 11 - RNA, 698 nt, synthetic construct
  source 1..698: mol_type = other RNA
gatatatgtctcgctccctttcgcttacagaaaggatttcttccgacagacgtgaccccagaaggctgccgagggagactttaggacaagaggcgttgttttttttcagcgtggccgcatttggaaaaggctgaaggtagaaaactctggaatggagaggctggcgactggagcaggatccaggggctttaaattttaacctgaatgcgatcgaatgcgcgactcccagaagagatagccaggcgtccgcccggccgctcaacccccgccaggactcggaatgccttgcttttggttcttttctacaaggatgtctgcgtggatatccctggacactcgataccccccggggacctgcttttcccgcagctccgcagacgcggcgggagggcgcgctggccccggcgctgctgggacagaggtgagcagcggggtggacattgaccgattcgacaacgttcgggctctaagggctcgggaagggacccggcattttaccctccagattcccaatctatttttaaaaataatgttcaaaacatatgggaaaaataagtgattgctccttttcatggtcacagattgtatttattttttcagactggttttccaacctaacgagggaatgggtagtgaatgttgttgttgttgttgttgtttatgttggttaatttcgaaataaaactgtttaactcgta

SEQ ID NO: 12 - RNA, 1341 nt, synthetic construct
  source 1..1341: mol_type = other RNA
agttatcacaactctgtacaaactgatacggtccctagcgggcaaaagggactggaaatgaacgtcgcggggttatcagcgccgatctgtcagcggcaactcgcgcggccgcaggctcgtccgctgcaggagccgtgatatatgtctcgctccctttcgcttacagaaaggatttcttccgacagacgtgaccccagaaggctgccgagggagactttaggacaagaggcgttgtttttgtgagaatgaatttttatactttaccccgaccccaccccctaccccccgcccaggtaaatccaagtaaatgattttctgaaggtcggtctcctagaaagcgtttaggctctggttttttgttttgtttctattttcgtgttttcccagttagggaaggaagaggagcctgtgcagagttgaactggggtggagaggtgccttgtaaagtatggtttccagagatggtgagacctcgtagctaagcgttgtttcctctgcacgtaactgtgagttgtgaacttgtggctcctgggggagcgggacagggcctggcagtgcgggggagggggcaaggaattcaggaagttggcgagaattcttcgtttttcattcttcgttgtaaattgcttctaaacacttctgaattttaaattgctaccgagaaggtccagaaaccgttctcatccaatttagtcacttttattacgttccactttgcctcccccaatcactgcggcggctcccagtttcagcgtggccgcatttggaaaaggctgaaggtagaaaactctggaatggagaggctggcgactggagcaggatccaggggctttaaattttaacctgaatgcgatcgaatgcgcgactcccagaagagatagccaggcgtccgcccggccgctcaacccccgccaggactcggaatgccttgcttttggttcttttctacaaggatgtctgcgtggatatccctggacactcgataccccccggggacctgcttttcccgcagctccgcagacgcggcgggagggcgcgctggccccggcgctgctgggacagaggtgagcagcggggtggacattgaccgattcgacaacgttcgggctctaagggctcgggaagggacccggcattttaccctccagattcccaatctatttttaaaaataatgttcaaaacatatgggaaaaataagtgattgctccttttcatggtcacagattgtatttattttttcagactggttttccaacctaacgagggaatgggtagtgaatgttgttgttgttgttgttgtttatgttggttaatttcgaaataaaactgtttaactcgta

SEQ ID NO: 13 - RNA, 637 nt, synthetic construct
  source 1..637: mol_type = other RNA
gaaaggatttcttccgacagacgtgaccccagaaggctgccgagggagactttaggacaagaggcgttgtttttttagggaaggaagaggagcctgtgcagagttgaactggggtggagaggtgccttgtaaagtatggtttccagagatggtgagacctcgtagctaagcgttgtttcctctgcacgtaacttttcagcgtggccgcatttggaaaaggctgaaggtagaaaactctggaatggagaggctggcgactggagcaggatccaggggctttaaattttaacctgaatgcgatcgaatgcgcgactcccagaagagatagccaggcgtccgcccggccgctcaacccccgccaggactcggaatgccttgcttttggttcttttcgtgagttccgaccaaatgtttaagtggactgggttttgcagaagctcatttgtaagcactgacggaaatgggttgtgggcatttgatttcctttgattccaacgttttgaaataatgtttctcggatttgctattcgaagcaattttctaaaaactcatatcttatttgctaaaaataaactctttggctcagtttgtgtccaggctgtgtacggcctccaaccctgagccccgaggagatcga

SEQ ID NO: 14 - RNA, 872 nt, synthetic construct
  source 1..872: mol_type = other RNA
gtccgctgcaggagccgtgatatatgtctcgctccctttcgcttacagaaaggatttcttccgacagacgtgaccccagaaggctgccgagggagactttaggacaagaggcgttgtttttttagggaaggaagaggagcctgtgcagagttgaactggggtggagaggtgccttgtaaagtatggtttccagagatggtgagacctcgtagctaagcgttgtttcctctgcacgtaactgtgagttgtgaacttgtggctcctgggggagcgggacagggcctggcagtgcgggggagggggcaaggaattcaggaagttggcgagaattcttcgtttttcattcttcgttgtaaattgcttctaaacacttctgaattttaaattgctaccgagaaggtccagaaaccgttctcatccaatttagtcacttttattacgttccactttgcctcccccaatcactgcggcggctcccagtttcagcgtggccgcatttggaaaaggctgaaggtagaaaactctggaatggagaggctggcgactggagcaggatccaggggctttaaattttaacctgaatgcgatcgaatgcgcgactcccagaagagatagccaggcgtccgcccggccgctcaacccccgccaggactcggaatgccttgcttttggttcttttcgtgagttccgaccaaatgtttaagtggactgggttttgcagaagctcatttgtaagcactgacggaaatgggttgtgggcatttgatttcctttgattccaacgttttgaaataatgtttctcggatttgctattcgaagcaattttctaaaaactcatatcttatttgctaaaaataaactctttggctca

SEQ ID NO: 15 - RNA, 72 nt, synthetic construct
  source 1..72: mol_type = other RNA
gctgtgggtcggaactgcgctcagcgagcagctgggcgctggaggtaaatccaagtaaatgattttctgaag

SEQ ID NO: 16 - RNA, 21 nt, synthetic construct
  source 1..21: mol_type = other RNA
gcgcattcgatcgcattcagg

SEQ ID NO: 17 - RNA, 21 nt, synthetic construct
  source 1..21: mol_type = other RNA
cctgaatgcgatcgaatgcgc

SEQ ID NO: 18 - RNA, 21 nt, synthetic construct
  source 1..21: mol_type = other RNA
ggagtcgcgcattcgatcgca

SEQ ID NO: 19 - RNA, 21 nt, synthetic construct
  source 1..21: mol_type = other RNA
tgcgatcgaatgcgcgactcc

SEQ ID NO: 20 - RNA, 25 nt, synthetic construct
  source 1..25: mol_type = other RNA
cagtttgaagatgggatcctagaca

SEQ ID NO: 21 - RNA, 25 nt, synthetic construct
  source 1..25: mol_type = other RNA
tgtctaggatcccatcttcaaactg

SEQ ID NO: 22 - RNA, 18 nt, synthetic construct
  source 1..18: mol_type = other RNA
ttctgggagtcgcgcatt

SEQ ID NO: 23 - RNA, 18 nt, synthetic construct
  source 1..18: mol_type = other RNA
gtggccgcatttggaaaa

SEQ ID NO: 24 - RNA, 20 nt, synthetic construct
  source 1..20: mol_type = other RNA
ttcccatgactccaacttcc

SEQ ID NO: 25 - RNA, 20 nt, synthetic construct
  source 1..20: mol_type = other RNA
cgcctatgtagagcccatct

SEQ ID NO: 26 - RNA, 19 nt, synthetic construct
  source 1..19: mol_type = other RNA
gctggctaacttgctgtgg

SEQ ID NO: 27 - RNA, 18 nt, synthetic construct
  source 1..18: mol_type = other RNA
gcccttcagctgcacttt

SEQ ID NO: 28 - RNA, 20 nt, synthetic construct
  source 1..20: mol_type = other RNA
ctgaacctacgagtacaccg

SEQ ID NO: 29 - RNA, 20 nt, synthetic construct
  source 1..20: mol_type = other RNA
ttaattctaggacgatgggc

SEQ ID NO: 30 - RNA, 20 nt, synthetic construct
  source 1..20: mol_type = other RNA
gtgtgatgtgagggatcctg

SEQ ID NO: 31 - RNA, 20 nt, synthetic construct
  source 1..20: mol_type = other RNA
tagtgtcacgaaggctgtgc

SEQ ID NO: 32 - RNA, 28 nt, synthetic construct
  source 1..28: mol_type = other RNA
ggagaagtttttgaagagggctgagaat

SEQ ID NO: 33 - RNA, 27 nt, synthetic construct
  source 1..27: mol_type = other RNA
cagacccacacaatacatgaagtgttg

SEQ ID NO: 34 - RNA, 24 nt, synthetic construct
  source 1..24: mol_type = other RNA
ggtgacaacagcagtatggacgac

SEQ ID NO: 35 - RNA, 20 nt, synthetic construct
  source 1..20: mol_type = other RNA
cagccagccatcaagcaggt

SEQ ID NO: 36 - RNA, 18 nt, synthetic construct
  source 1..18: mol_type = other RNA
tggacctcatggcccaca

SEQ ID NO: 37 - RNA, 20 nt, synthetic construct
  source 1..20: mol_type = other RNA
tcaaggggtctacatggcaa

SEQ ID NO: 38 - RNA, 18 nt, synthetic construct
  source 1..18: mol_type = other RNA
ggcatggtcaactgcaac

SEQ ID NO: 39 - RNA, 20 nt, synthetic construct
  source 1..20: mol_type = other RNA
cagcactggccatacttgac

SEQ ID NO: 40 - RNA, 20 nt, synthetic construct
  source 1..20: mol_type = other RNA
cctcgttaggttggaaaacc

SEQ ID NO: 41 - RNA, 20 nt, synthetic construct
  source 1..20: mol_type = other RNA
ctcgtacagttgattcagac

SEQ ID NO: 42 - RNA, 20 nt, synthetic construct
  source 1..20: mol_type = other RNA
tatcgagtgtccagggatat

SEQ ID NO: 43 - RNA, 20 nt, synthetic construct
  source 1..20: mol_type = other RNA
atctggagggtaaaatgccg

SEQ ID NO: 44 - RNA, 20 nt, synthetic construct
  source 1..20: mol_type = other RNA
attccagagttttctacctt

SEQ ID NO: 45 - RNA, 20 nt, synthetic construct
  source 1..20: mol_type = other RNA
aaaagcaaggcattccgagt

SEQ ID NO: 46 - RNA, 20 nt, synthetic construct
  source 1..20: mol_type = other RNA
caccatctctggaaaccata

SEQ ID NO: 47 - RNA, 20 nt, synthetic construct
  source 1..20: mol_type = other RNA
accgttctcatccaatttag

SEQ ID NO: 48 - RNA, 20 nt, synthetic construct
  source 1..20: mol_type = other RNA
agaaactctccagggacatc

SEQ ID NO: 49 - RNA, 20 nt, synthetic construct
  source 1..20: mol_type = other RNA
aggacaagaggcgttgtttt

SEQ ID NO: 50 - RNA, 20 nt, synthetic construct
  source 1..20: mol_type = other RNA
ggcaaaaagcgcatttgctg

SEQ ID NO: 51 - RNA, 20 nt, synthetic construct
  source 1..20: mol_type = other RNA
caaaggtcagctggggaatg

(19) United States
(12) Patent Application Publication
(10) Pub. No.: US 2024/0067966 A1
(43) Pub. Date: Feb. 29, 2024

(54) GATA6-AS1 lncRNA FOR USE IN THERAPY

(71) Applicants: Tel Hashomer Medical Research Infrastructure and Services Ltd., Ramat Gan (IL); Ramot At Tel-Aviv University Ltd., Tel Aviv (IL)

(72) Inventors: Yael HABERMAN ZIV, Tel Aviv (IL); Katya Elinoy SOSNOVSKI, Petach-Tikva (IL); Tzipi BRAUN, Giv'at Shmuel (IL)

(21) Appl. No.: 18/310,044

(22) Filed: May 1, 2023

Related U.S. Application Data
(60) Provisional application No. 63/363,817, filed on Apr. 29, 2022.

(51) Int. Cl. C12N 15/113; A61K 45/06; A61P 29/00

(52) U.S. Cl. CPC C12N 15/113; A61K 45/06; A61P 29/00; G01N 33/6893

(57) ABSTRACT

The present invention provides methods of treating or preventing chronic intestinal inflammation and/or inflammatory bowel disease (IBD) comprising administering GATA6-AS1 lncRNA to a patient in need thereof.

The contents of the electronic sequence listing (Sequence_Listing_GATA6-AS1 lncRNA FOR THERAPY.xml; Size: 61,445 bytes; and Date of Creation: Apr. 27, 2023) is herein incorporated by reference in its entirety.

### TECHNOLOGICAL FIELD

The present disclosure relates to therapeutic compositions and methods for treating inflammatory gut diseases.

### BACKGROUND

Ulcerative colitis (UC) and Crohn Disease (CD) are prevalent intestinal inflammatory manifestations in Westernized countries affecting ˜1-4:1000 [1]. Patients often require life-long therapy, but currently available medications may not be effective or lose their effectiveness over time, with sustained remission seen in <50% of cases. Celiac disease is another prevalent enteropathy, affecting 1-4:100, triggered by dietary gluten [2]. Proper epithelial metabolic and barrier functions are key to facilitate mucosal repair and healing in all three diseases. Moreover, mucosal healing is an important predictor of long-term remission in IBD [3,4]. Yet there are no epithelial directed therapies [5].

Long non-coding RNAs (LncRNAs) are defined as RNA molecules greater than 200 nucleotides in length that have low protein-coding potential. They are mostly transcribed by RNA polymerase II, capped, polyadenylated, and spliced similarly to other mRNA [6], but are not predicted to encode for functional proteins.

LncRNAs are known to play key roles in transcription regulation, metabolic pathways, intestinal barrier functions, and have also been implicated in human disease [7]. A widespread dysregulation of lncRNAs was identified in an ileal CD cohort [8], linking these lncRNAs to CD pathogenesis. Predicting Response to Standardized Pediatric Colitis Therapy (PROTECT) is a large prospective UC inception cohort that examined factors associated with pathogenesis and responses to standardized therapy. By characterization of rectal protein-coding genes expression, Haberman et al (2019) [9] reported a robust reduction in epithelial mitochondrial genes and energy production pathways in UC. Hyams et al (2019 and 2021) [10, 11] defined genes and pathways linked to UC course.

### GENERAL DESCRIPTION

In a first of its aspects, the present invention provides a method of treating or preventing chronic intestinal inflammation and/or inflammatory bowel disease (IBD) in an individual in need thereof comprising administration of GATA6-AS1 lncRNA to said individual.

In another aspect, the present invention provides a pharmaceutical composition comprising GATA6-AS1 lncRNA, and a pharmaceutically acceptable carrier.

In one embodiment, the pharmaceutical composition is for treating or preventing chronic intestinal inflammation and/or inflammatory bowel disease (IBD) in an individual, and/or for increasing mitochondrial membrane potential and/or mitochondrial respiration in intestinal epithelial cells.

In one embodiment, the individual has been diagnosed by a method that comprises measuring GATA6-AS1 lncRNA in the individual, detecting a reduced level of GATA6-AS1 lncRNA as compared with a reference level, and thereby providing a positive diagnosis for chronic intestinal inflammation and/or IBD in the individual.

In one embodiment, the administration of the GATA6-AS1 lncRNA or functional fragment thereof to the individual is following diagnosis of chronic intestinal inflammation and/or IBD in the individual using a method that comprises measuring GATA6-AS1 lncRNA in the individual, detecting reduced level of GATA6-AS1 lncRNA as compared with a reference level, and thereby providing a positive diagnosis for chronic intestinal inflammation and/or IBD in the individual.

In one embodiment, administration of the GATA6-AS1 lncRNA to said individual improves mitochondrial function (e.g., increasing mitochondrial membrane potential and/or mitochondrial respiration) in the intestinal epithelia.

In one embodiment, the individual is a mammal.

In one embodiment, the mammal is a human.

In one embodiment, said GATA6-AS1 lncRNA comprises or consists of a sequence that is about 90-100% identical to any one of SEQ ID No. 1-15.

In one embodiment, said GATA6-AS1 lncRNA is packaged in or attached to a delivery system. In some embodiments, said delivery system is selected from a group consisting of a viral-based delivery system, an exosome, a polymer-based delivery system, a lipid-based delivery system, and a conjugate-based delivery system.

In one embodiment, said GATA6-AS1 lncRNA is administered by rectal administration, oral administration, or by injection (e.g., intramuscular, or intravenous injection).

In another aspect, the present invention provides a method of treating or preventing chronic intestinal inflammation and/or inflammatory bowel disease (IBD) in an individual in need thereof comprising administration of an expression vector (e.g., a viral expression vector) encoding GATA6-AS1 lncRNA to said individual.

The invention also provides a pharmaceutical composition comprising an expression vector (e.g., a viral expression vector) encoding GATA6-AS1 lncRNA, and a pharmaceutically acceptable carrier.

In another aspect, the present invention provides a method of increasing mitochondrial membrane potential and/or mitochondrial respiration in intestinal epithelial cells comprising administration of GATA6-AS1 lncRNA.

In another aspect, the present invention provides a kit comprising (i) means for determining the level of GATA6-AS1 lncRNA in an individual having or suspected of having or being at risk of developing chronic intestinal inflammation and/or IBD; and (ii) a pharmaceutical composition according to the invention.

In some embodiments, the chronic intestinal inflammation or IBD is Crohn's disease (CD), ulcerative colitis (UC) or celiac.

In some embodiments, the treatment is administered in combination with a TGM2 antagonist.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

To better understand the subject matter that is disclosed herein and to exemplify how it may be carried out, embodiments will now be described, by way of non-limiting example only, with reference to the accompanying drawings, in which:

FIG. 1A shows a chart of Epithelia where the size and color of the dots are proportional to the percent expressing the gene and the normalized expression level.

FIG. 1B shows a chart of Immune cells where the size and color of the dots are proportional to the percent expressing the gene and the normalized expression level.

FIG. 1C shows a chart of Stroma where the size and color of the dots are proportional to the percent expressing the gene and the normalized expression level.

FIG. 1D-1F show a graph of GATA6-AS1 mRNA expression in cases and controls of bulk UC (PROTECT, RISK) and secondary analyses of isolated epithelia [13]. Plot with central line indicating median and additional lines representing upper and lower quartile.

FIG. 1G-1I show a graph of GATA6-AS1 mRNA expression in cases and controls of CD (SOURCE, RISK) and secondary analyses of isolated epithelia [13]. Plot with central line indicating median and additional lines representing upper and lower quartile.

FIGS. 1J and 1K show a graph of GATA6-AS1 mRNA expression in cases and controls of bulk celiac biopsies (SEEM, and celiac cohort [14]). Plot with central line indicating median and additional lines representing upper and lower quartile.

FIG. 1L shows a graph of GATA6-AS1 mRNA expression with UC cases correlation with clinical [Ulcerative Colitis Activity Index (PUCAI)]. GATA6-AS1 levels in controls are given as a reference for normal signal.

FIG. 1M shows a graph of GATA6-AS1 mRNA expression with UC cases correlation with combined clinical-endoscopic (Total Mayo). GATA6-AS1 levels in controls are given as a reference for normal signal.

FIG. 1N shows a graph of GATA6-AS1 mRNA expression with UC cases, stratified by endoscopic disease severity (Endoscopic Mayo score). GATA6-AS1 levels in controls are given as a reference for normal signal.

FIG. 1O shows a graph of GATA6-AS1 mRNA expression with UC cases by disease outcome (W4R). GATA6-AS1 levels in controls are given as a reference for normal signal.

FIG. 1P shows a graph of GATA6-AS1 mRNA expression with UC cases by disease outcome (W52SFR). GATA6-AS1 levels in controls are given as a reference for normal signal.

FIG. 1Q shows a graph of GATA6-AS1 expressed in Caco2 and HT29 human-derived intestinal tissue culture model, whereby it is distributed between nucleus and cytoplasm in Caco2 cells as indicated by both fluorescent in situ hybridization (FISH) and Hoechst staining, and its expression is reduced upon inflammatory triggers (100 ng/ml LPS plus 40 ng/ml IFNγ) and hypoxia (100 μM DFO), with induction of IL8 (CXCL8) in similar conditions in HT29. (Magnification—×63 oil; Scalebar—10 μM).

FIG. 1R-1U show graphs of GATA6-AS1 expressed in patient-derived ileum and colon organoid culture with reduction upon 25 ng/ml IFNγ, plus 20 ng/ml TNFα treatment.

FIG. 1V shows GATA6-AS1 transcripts per million (TPM) in controls and non-inflamed ileum of Crohn's Disease (CD) patients in the RISK cohort.

FIG. 1W shows GATA6-AS1 transcripts per million (TPM) in controls and non-inflamed colon epithelia of Ulcerative Colitis (UC) patients.

FIG. 1X shows GATA6-AS1 transcripts per million (TPM) in controls and non-inflamed ileum epithelia of Crohn's Disease (CD) patients. T-test or Spearman's correlation with coefficients (r) are shown. All 2-sided P<0.05, **P<0.01, ***P<0.001, ****P<0.0001.

FIG. 2A shows a schematic representation of GATA6-S1 and protein complex pull-down experiments. LncRNA:protein interactions were identified by tiling antisense-oligos designed to capture GATA6-AS1, those were separated into two independent pools (6 oligos), which served as internal controls for each other and real RNA-dependent signals would be present in both.

FIG. 2B shows analysis of the GATA6-AS1 interactome by mass spectrometry identified 285 proteins (T-test looking at higher binding with GATA6-AS1 oligos vs. controls with delta LFQ≥0.1 and FDR ≤0.05). Functional enrichment analysis indicated enrichment to epithelial and mitochondrial functions (Toppgene/Toppcluster functional annotation enrichment software).

FIG. 2C-2G show GATA6-AS1 silencing was achieved using two GATA6-AS1 specific and independent shRNA (sh1, sh2) in Caco2 cells, which were compared with scrambled nonspecific shRNA (Ct1) using qRT-PCR (normalized to GAPDH and to levels in controls). This resulted in reduction of OTC, MT-C02, DECR1, and LGR5 mRNA expression. Bar graphs with individual RQ values are shown.

FIG. 2H shows representative Western blots using specific antibodies in GATA6-AS1 knockdown indicated reduction of DECR1, MT-CO2, with induction of TGM2 and EPHX protein levels. GAPDH is used for loading control.

FIG. 2I shows mitochondrial ROS levels and apoptosis were measured using MitoSOX at baseline.

FIG. 2J shows mitochondrial ROS levels and apoptosis were measured using MitoSOX after treatment with 500 μM of H₂O₂ for 3 hr) and Annexin V.

FIG. 2K shows mitochondrial ROS levels and apoptosis were measured using MitoSOX at baseline.

FIG. 2L shows mitochondrial ROS levels and apoptosis were measured using MitoSOX after treatment with 500 μM of H²0² for 3 hr) staining in GATA6-AS1 knockdown.

FIG. 2M shows JC1 staining and FACS analysis were used to define the mitochondrial membrane potential with representative FACS data

FIG. 2N shows a bar graph showing the average of 3 experiments. Individual values are shown in the graph with their mean. All 2-sided paired T-test, P<0.05, **P<0.01, ***P<0.001, ****P<0.0001.

FIG. 3A shows schematic representation of four mitochondrial complexes of the Electron Transport Chain (I, II, III, IV) and ATP synthase (complex V), located in the inner mitochondrial membrane and modulators included in the Seahorse XF Cell Mito Stress Test.

FIG. 3B shows XF Cell Mito Stress Test (see methods) was used to measure Oxygen Consumption Rate (OCR), under basal conditions.

FIG. 3C shows XF Cell Mito Stress Test (see methods) was used to measure Oxygen Consumption Rate (OCR) under exposure to DFO+LPS+INFγ (C)

FIG. 3D shows XF Cell Mito Stress Test (see methods) was used to measure Oxygen Consumption Rate (OCR), after sequential injections of oligomycin, FCCP and rotenone/antimycin A in GATA6-AS1 knockdown and control Caco2 cells, and changes in basal respiration.

FIG. 3E shows XF Cell Mito Stress Test (see methods) was used to measure Oxygen Consumption Rate (OCR) with maximal respiration (E).

FIG. 3F shows XF Cell Mito Stress Test (see methods) was used to measure Oxygen Consumption Rate (OCR) where ATP production were calculated (Grey—Ct1 shRNA; Light green—GATA6-AS1 shRNA1; Light blue—GATA6-AS1 shRNA2).

FIG. 3G shows Untargeted metabolomic with a pre-defined library of 170 metabolites were used to capture cellular metabolites and metabolites secreted to the media (see methods) in GATA6-AS1 knockdown and control cells, untreated or treated with 100 μM DFO+100 ng/ml LPS+40 ng/ml IFNγ. PCoA Plot of the extracted metabolites is presented indicating differences by both GATA6-AS1 expression and by treatment in cells; Color codes: Dark blue—Baseline, control (Ct1); Dark purple—Baseline, GATA6-AS1 ShRNA1; Dark green—Baseline, GATA6-AS1 ShRNA2; Light blue—treated with DFO+LPS+IFNγ, control (Ct1); Light purple—treated with DFO+LPS+IFNγ, GATA6-AS1 ShRNA1; Light green—treated with DFO+LPS+IFNγ, GATA6-AS1 ShRNA2.

FIG. 3H shows Untargeted metabolomic with a pre-defined library of 170 metabolites were used to capture cellular metabolites and metabolites secreted to the media (see methods) in GATA6-AS1 knockdown and control cells, untreated or treated with 100 μM DFO+100 ng/ml LPS+40 ng/ml IFNγ. PCoA Plot of the extracted metabolites is presented indicating differences by both GATA6-AS1 expression and by treatment in media; Color codes: Dark blue—Baseline, control (Ct1); Dark purple—Baseline, GATA6-AS1 ShRNA1; Dark green—Baseline, GATA6-AS1 ShRNA2; Light blue—treated with DFO+LPS+IFNγ, control (Ct1); Light purple—treated with DFO+LPS+IFNγ, GATA6-AS1 ShRNA1; Light green—treated with DFO+LPS+IFNγ, GATA6-AS1 ShRNA2.

FIG. 3I shows XF Cell Mito Stress Test was used to measure OCR (as in FIG. 3A) in GATA6-AS1 knockdown and controls cells transiently transfected with TGM2 or control siRNA, to test if reduction of TGM2 can rescue GATA6-AS1 knockdown effect of cellular respiration; Color codes: Grey—control (Ct1) shRNA; Light green—GATA6-AS1 shRNA1; Light blue—GATA6-AS1 shRNA2; Light purple—control (Ct1) shRNA+siTGM2; Light green encircled in purple-GATA6-AS1 shRNA1+siTGM2; Light blue encircled in purple—GATA6-AS1 shRNA2+siTGM2.

FIG. 3J shows XF Cell Mito Stress Test was used to measure OCR (as in FIG. 3A) in GATA6-AS1 knockdown and controls cells transiently transfected with TGM2 or control siRNA, to test changes in basal respiration; Color codes: Grey—control (Ct1) shRNA; Light green—GATA6-AS1 shRNA1; Light blue—GATA6-AS1 shRNA2; Light purple—control (Ct1) shRNA+siTGM2; Light green encircled in purple—GATA6-AS1 shRNA1+siTGM2; Light blue encircled in purple—GATA6-AS1 shRNA2+siTGM2.

FIG. 3K shows XF Cell Mito Stress Test was used to measure OCR (as in FIG. 3A) in GATA6-AS1 knockdown and controls cells transiently transfected with TGM2 or control siRNA, to test maximal respiration; Color codes: Grey—control (Ct1) shRNA; Light green—GATA6-AS1 shRNA1; Light blue—GATA6-AS1 shRNA2; Light purple—control (Ct1) shRNA+siTGM2; Light green encircled in purple—GATA6-AS1 shRNA1+siTGM2; Light blue encircled in purple—GATA6-AS1 shRNA2+siTGM2.

FIG. 3L shows XF Cell Mito Stress Test was used to measure OCR (as in FIG. 3A) in GATA6-AS1 knockdown and controls cells transiently transfected with TGM2 or control siRNA, to test ATP production; Color codes: Grey—control (Ct1) shRNA; Light green—GATA6-AS1 shRNA1; Light blue—GATA6-AS1 shRNA2; Light purple—control (Ct1) shRNA+siTGM2; Light green encircled in purple—GATA6-AS1 shRNA1+siTGM2; Light blue encircled in purple—GATA6-AS1 shRNA2+siTGM2.

FIG. 3M shows extracellular acidification rate (ECAR) was measured, whereby the non-glycolytic acidification was calculated and found to be different between GATA6-AS1 knockdown and control cells, and to be rescued by reduction of TGM2. Color codes as for FIG. 3L.

FIG. 3N shows concluding scheme for GATA6-AS1 regulation of epithelial cellular mitochondrial functions. GATA6-AS1 binds a complex that includes TGM2 (1). GATA6-AS1 silencing resulted in TGM2 induction similarly to the induction seen in patients (1). This together caused attenuation of mitochondrial membrane potential (2), higher production of mitochondrial ROS (linked with inflammation, 3), reduction of TCA metabolites (4), and reduced cellular respiration (5). Individual values are shown in the graphs with their mean. All 2-sided paired T test, P<0.05, **P<0.01. Scheme was created with biorender.com.

### DETAILED DESCRIPTION OF EMBODIMENTS

The present invention is based in part on the discovery that GATA6-AS1 lncRNA is reduced in several inflammatory conditions of the gastrointestinal tract.

The present invention thus provides the use of GATA6-AS1, optionally in the form of lncRNA-lipid nanoparticle (lncRNA-LNPs) for correcting the reduced levels of GATA6-AS1 in epithelia and thereby to enable proper epithelial mitochondrial functions, proliferation, and regeneration to ensure mucosal healing as potential intervention in inflammatory conditions of the gastrointestinal tract.

A comprehensive atlas of lncRNAs expressed along the gastrointestinal (GI) tract (small and large intestine) was generated and lncRNAs whose expression was dysregulated in celiac disease, Crohn disease (CD), and ulcerative colitis (UC) was identified using an independent test and validation cohorts (673 mucosal mRNAseq samples).

The inventors utilized the large, well-designed, and characterized PROTECT UC cohort, and CD and celiac cohorts of treatment-naïve patients, free from confounding variables of previous therapy that may affect gene expression. Other cohorts including the large RISK and publicly available celiac cohorts as well as published intestinal epithelial transcriptomics and single cell dataset provided independent validation. This enabled the inventors to prioritize and then verify in cellular assays the informatic analyses suggesting a role for GATA6-AS1 in mitochondrial function. Complementary approaches were used combining transcriptomics, metabolomics, proteomics, and specific mitochondrial functional assays to cross-validate the results.

Several methodologies were employed to prioritize functional lncRNAs including lncRNA associations with disease, severity, outcome, as well as WGCNA as a measure to identify central lncRNA within modules linked with clinical features and to infer functionality using the better annotated co-expressed protein-coding genes. This highlighted 37 lncRNAs and 817 protein coding genes including reduction of GATA6-AS1 and induction of TGM2 in celiac disease, CD and UC.

GATA6-AS1 lncRNA was reduced in all three diseases and its interactome included TGM2 (an autoantigen in celiac disease) which was increased in all 3 diseases, with an overall enrichment of the interactome for mitochondrial functions. GATA6-AS1 silencing resulted in induction of TGM2, attenuated mitochondrial membrane potential, reduced mitochondrial respiration and effects on cellular metabolomics with reduction of tricarboxylic acid (TCA) cycle metabolites, changes that are relevant to disease.

As evidenced in the Examples below, reduction of GATA6-AS1 was demonstrated in epithelial and mucosal biopsies from UC, CD, and celiac, in non-clinical inflamed samples, and with inflammatory triggers in different epithelial model systems (cell lines and patient-derived organoid), which indicate that inflammation and potentially other factors contribute to the regulation of GATA6-AS1 expression. Mimicking GATA6-AS1 reduction in model system led to mitochondrial ROS accumulation and induction of apoptosis. Additionally, GATA6-AS1 silencing resulted in an induction of TGM2, and in attenuation of mitochondrial membrane potential, reduction of mitochondrial respiration, and a robust effect on cellular metabolomics with reduction of several tricarboxylic acid (TCA) cycle metabolites including α-ketoglutarate (aKG) relevant to disease. TGM2, also known as tissue transglutaminase and the autoantigen implicated in celiac disease, has also been shown to regulate Ca2+ flux to mitochondria and TGM2 overexpressing cells exhibited a low basal OCR as shown in the Examples upon GATA6-AS1 knockdown. Reducing TGM2 levels in GATA6-AS1 knockdown rescued mitochondrial respiration, indicating that GATA6-AS1 regulation of mitochondrial function may occur through GATA6-AS1 interaction and regulation of TGM2 expression.

The present invention therefore provides a method for diagnosing inflammatory gut diseases comprising measuring the levels of GATA6-AS1 lncRNA, whereby a reduction in GATA6-AS1 lncRNA levels can be used as a biomarker for these diseases, as well as a biomarker indicating the severity of the disease.

The present invention also provides GATA6-AS1 lncRNA as a target for therapeutic RNA-based interventions to boost epithelial mitochondrial metabolic functions and renewal to enable mucosal healing and improve outcome.

In addition to GATA6-AS1, prioritized lncRNAs that were found to be dysregulated in inflammatory gastrointestinal conditions include Morrbid lncRNA (also known as MIR4435-2HG), which was up-regulated, INFG-AS1 (also termed NeST), CEROX1 (also known as RP11-161M6.2), and LINC01272. H19, was also prioritized in the analyses, as well as reduction of ANRIL (CDKN2B-AS1). Microbial organisms and products affect host mucosal barrier function and aberrant immune responses to commensal microbes likely contribute to gut inflammation. Significant positive associations were identified between GATA6-AS1 and CDKN2B-AS1 expression and Lachnospiraceae Blautia (ASV09918), as well as a negative association between GATA6-AS1 and Enterococcus (ASV13969), and positive association between LINGO 1272 and Streptococcus (AS V12252).

In a first of its aspects, the present invention provides a method of treating or preventing chronic intestinal inflammation and/or inflammatory bowel disease (IBD) in an individual in need thereof comprising administration of GATA6-AS1 lncRNA or a functional fragment thereof to said individual.

As used herein, “treating” an individual suffering from chronic intestinal inflammation and/or inflammatory bowel disease (IBD) means administration to an individual by any suitable dosage regimen, procedure and/or administration route of a composition in accordance with the present invention, with the object of achieving a desirable clinical/medical end-point, including but not limited to, healing, ameliorating, stopping or slowing progression, reversing, and/or reducing the rate of advancement of intestinal inflammation and/or inflammatory bowel disease (IBD) such that symptoms of a given disorder are ameliorated.

As used herein, “chronic intestinal inflammation and/or inflammatory bowel disease (IBD)” encompass, but are not limited to, ulcerative colitis (UC), Crohn's disease and celiac.

The terms “patient”, “individual” or “subject” are used interchangeably herein and refer to vertebrates, specifically to mammals (e.g., humans, simians, pets such as cats, dogs, guinea pigs, mice, rats, rabbits, ferret and the like, farm and sport animals such as horses, donkeys, chicken, turkey, alpaca, pigs, cows, goats, sheep and the like). In one embodiment said individual is a human.

The term “nucleic acid” or “nucleic acid molecule” as herein defined refers to a polymer of nucleotides, which may be either single- or double-stranded, which is a polynucleotide such as deoxyribonucleic acid (DNA), or ribonucleic acid (RNA), e.g., long non-coding RNA. The terms should also be understood to include, as equivalents, analogs of either RNA or DNA made from nucleotide analogs, and, as applicable to the embodiment being described, single-stranded (such as sense or antisense) and double-stranded polynucleotides.

The term encompasses sequences that include any of the known base analogs of RNA.

As used herein the term “long non-coding RNA (lncRNA)” refers to non-coding RNA molecules containing about 200 or more nucleotides that are not translated to a protein.

“GATA6-AS1” is a lncRNA divergently transcribed from the GATA6 locus.

The compositions useful in the methods of the invention can include all or part of the GATA6-AS1 lncRNA, including a functional fragment thereof. As used herein the term “functional fragment thereof” refers to a nucleic acid sequence that forms a part of the GATA6-AS1 lncRNA and maintains the physiological effects of the full length GATA6-AS1 lncRNA, e.g., its function in increasing mitochondrial membrane potential and/or mitochondrial respiration, as measured for example by the assays specified in the Examples.

The full genomic sequence of GATA6-AS1 is shown below and denoted as SEQ ID NO: 1:

full length-chr18:22166889-22169894
CCTGGTTTTCTGTCTGAATCAACTGTACGAGTTAAACAGTTTTATTTCGA
AATTAACCAACATAAACAACAACAACAACAACAACATTCACTACCCATTC
CCTCGTTAGGTTGGAAAACCAGTCTGAAAAAATAAATACAATCTGTGACC
ATGAAAAGGAGCAATCACTTATTTTTCCCATATGTTTTGAACATTATTTT
TAAAAATAGATTGGGAATCTGGAGGGTAAAATGCCGGGTCCCTTCCCGAG
CCCTTAGAGCCCGAACGTTGTCGAATCGGTCAATGTCCACCCCGCTGCTC
ACCTCTGTCCCAGCAGCGCCGGGGCCAGCGCGCCCTCCCGCCGCGTCTGC
GGAGCTGCGGGAAAAGCAGGTCCCCGGGGGGTATCGAGTGTCCAGGGATA
TCCACGCAGACATCCTTGTACTTGCAAATACAAAGAAACACACAGGACAG
AGATCAGGCTCGCGCCAGGGCTCGATCTCCTCGGGGCTCAGGGTTGGAGG
CCGTACACAGCCTGGACACAAACTGAGCCAAAGAGTTTATTTTTAGCAAA
TAAGATATGAGTTTTTAGAAAATTGCTTCGAATAGCAAATCCGAGAAACA
TTATTTCAAAACGTTGGAATCAAAGGAAATCAAATGCCCACAACCCATTT
CCGTCAGTGCTTACAAATGAGCTTCTGCAAAACCCAGTCCACTTAAACAT
TTGGTCGGAACTCACGAAAAGAACCAAAAGCAAGGCATTCCGAGTCCTGG
CGGGGGTTGAGCGGCCGGGCGGACGCCTGGCTATCTCTTCTGGGAGTCGC
GCATTCGATCGCATTCAGGTTAAAATTTAAAGCCCCTGGATCCTGCTCCA
GTCGCCAGCCTCTCCATTCCAGAGTTTTCTACCTTCAGCCTTTTCCAAAT
GCGGCCACGCTGAAACTGGGAGCCGCCGCAGTGATTGGGGGAGGCAAAGT
GGAACGTAATAAAAGTGACTAAATTGGATGAGAACGGTTTCTGGACCTTC
TCGGTAGCAATTTAAAATTCAGAAGTGTTTAGAAGCAATTTACAACGAAG
AATGAAAAACGAAGAATTCTCGCCAACTTCCTGAATTCCTTGCCCCCTCC
CCCGCACTGCCAGGCCCTGTCCCGCTCCCCCAGGAGCCACAAGTTCACAA
CTCACAGTTACGTGCAGAGGAAACAACGCTTAGCTACGAGGTCTCACCAT
CTCTGGAAACCATACTTTACAAGGCACCTCTCCACCCCAGTTCAACTCTG
CACAGGCTCCTCTTCCTTCCCTAACTGGGAAAACACGAAAATAGAAACAA
AACAAAAAACCAGAGCCTAAACGCTTTCTAGGAGACCGACCTTCAGAAAA
TCATTTACTTGGATTTACCTGGGCGGGGGGTAGGGGGTGGGGTCGGGGTA
AAGTATAAAAATTCATTCTCACAAAAACAACGCCTCTTGTCCTAAAGTCT
CCCTCGGCAGCCTTCTGGGGTCACGTCTGTCGGAAGAAATCCTTTCTGTA
AGCGAAAGGGAGCGAGACATATATCACGGCTCCTGCAGCGGACGAGCCTG
CGGCCGCGCGAGTTGCCGCTGACAGATCGGCGCTGATAACCCCGCGACGT
TCATTTCCAGTCCCTTTTGCCCGCTAGGGACCGTATCAGTTTGTACAGAG
TTGTGATAACTGTTTGGAGGGAGCGAAGAGGGGGTGTGTATATGAACGTG
GGGGTGGGGAAGCAGCATTTAGCGCTGGGTCCAGGGAAGGTGACACCCCC
TCTTCGCAGTGACTTATCTGTGACTTACCTGAAACTCTCCAGGGAAATAT
CAGAAACTCTCCAGGGACATCAAAAGTTGGAGAGCGTCCTCGGACACGAC
TGATGTGGAAGCCCTTTTCCATTCTGCGTACCCCATAGACTACCTTTCCG
TACATGACGACCCGAGTTAAAGTTCCCAAAGGTCAGCTGGGGAATGTTGT
CTTGAGGTTCCCGAAACCACCACGACCTGAGCCGTAGCATCCCGAGATAG
GGTCGGAGAAAGTTTAAGGTCGGTCTCACACAACTTCAGGGCAAAAAGCG
CATTTGCTGTGAAGGGGCTAGGCGGGGGTTGGGGGGCGGGGGGGCGCAGA
GCACGTTCCCCCTTCCTTCTGGGGGCGTGCTGACCCAAGGTCTGGAGCGC
CCCTCCCGCCGCGGCGGCGGCGCGGGACTGTGCACTGCCAACTCCTCCCG
TGCCAAGGCTCCCTCCCCCTCCCTCGTGTGGGTGTGTGTGTGCGACTGCG
GGAGCGGGAGGGTGCACCGCCGCTGGATGGGTGCGGGTCGCTAGCCAGGT
CAGGCGTTCTGTGGCCTCCGCCCCTCCCCCGGGGTCCCTGGGCTCGGTGC
CCCCCGGGTGGACTCGCCCCCACTCCGGGGACAGGGCTTGCGGCTCAGCC
CACGCCCGCAGGCAGCGCGGCTCTCATTGTCTGCCTGGGCGGCGCGCTCC
CCTCCCCCCTCGCCGTCCCCTCCCCACCCTCTTTTCTCTCCTCCCCTCGA
TCCCTCCTCCTCCTCTTCACCTCCAGCGCCCAGCTGCTCGCTGAGCGCAG
TTCCGACCCACAGCCTGGCACCCTTCGGCGAGCGCTGTTTGTTTAGGGCT
CGGTGAGTCCAATCAGGAGCCCAGGCTGCAGTTTTCCGGCAGAGCAGTAA
GAGGCGCCTCCTCTCTCCTTTTTATTCACCAGCAGCGCGGCGCAGACCCC
GGACTCGCGCTCGCCCGCTGGCGCCCTCGGCTTCTCTCCGCGCCTGGGAG
CACCCTCCGCCGCGGCCGTTCTCCATGCGCAGCGCCCGCCCGAGGTTCGG
CTGCTTCCTCCTCGCGCGATCTCCCCGCTTTCCTCCCCTCCACCCCTACT
CGTCGCGGGCCCCTGGCCTCCGGCTCTCCGGCCCTCTCCAGGCCGCGGTC
GGCTCCGACGGTGTTTCCCTCCTTCCCTCCGGGCGGGCTGAGGCGGCTCC
CAGGCTCCCGGGACACCGCGGACCAACTTCTAGTCTCTTTTTCTCTCCTC
GAAGCG

In some embodiments, the sequence of the GATA6-AS1 lncRNA is or comprises any one of the following RNA transcripts denoted respectively as SEQ ID Nos 2-15:

SEQ ID NO: 2:
AAACAGTTATCACAACTCTGTACAAACTGATACGGTCCCTAGCGGGCAAAAGGG
ACTGGAAATGAACGTCGCGGGGTTATCAGCGCCGATCTGTCAGCGGCAACTCGC
GCGGCCGCAGGCTCGTCCGCTGCAGGAGCCGTGATATATGTCTCGCTCCCTTTCG
CTTACAGAAAGGATTTCTTCCGACAGACGTGACCCCAGAAGGCTGCCGAGGGAG
ACTTTAGGACAAGAGGCGTTGTTTTTTTAGGGAAGGAAGAGGAGCCTGTGCAGA
GTTGAACTGGGGTGGAGAGGTGCCTTGTAAAGTATGGTTTCCAGAGATGGTGAG
ACCTCGTAGCTAAGCGTTGTTTCCTCTGCACGTAACTTTTCAGCGTGGCCGCATTT
GGAAAAGGCTGAAGGTAGAAAACTCTGGAATGGAGAGGCTGGCGACTGGAGCA
GGATCCAGGGGCTTTAAATTTTAACCTGAATGCGATCGAATGCGCGACTCCCAGA
AGAGATAGCCAGGCGTCCGCCCGGCCGCTCAACCCCCGCCAGGACTCGGAATGC
CTTGCTTTTGGTTCTTTTCTACAAGGATGTCTGCGTGGATATCCCTGGACACTCGA
TACCCCCCGGGGACCTGCTTTTCCCGCAGCTCCGCAGACGCGGCGGGAGGGCGC
GCTGGCCCCGGCGCTGCTGGGACAGAGGTGAGCAGCGGGGTGGACATTGACCGA
TTCGACAACGTTCGGGCTCTAAGGGCTCGGGAAGGGACCCGGCATTTTACCCTCC
AGATTCCCAATCTATTTTTAAAAATAATGTTCAAAACATATGGGAAAAATAAGTG
ATTGCTCCTTTTCATGGTCACAGATTGTATTTATTTTTTCAGACTGGTTTTCCAACC
TAACGAGGGAATGGGTAGTGAATGTTGTTGTTGTTGTTGTTGTTTATGTTGGTTAA
TTTCGAAATAAAACTGTTTAACTCGTACAGTTGATTCAGACAGAAAACCA
SEQ ID NO: 3:
AAACAGTTATCACAACTCTGTACAAACTGATACGGTCCCTAGCGGGCAAAAGGG
ACTGGAAATGAACGTCGCGGGGTTATCAGCGCCGATCTGTCAGCGGCAACTCGC
GCGGCCGCAGGCTCGTCCGCTGCAGGAGCCGTGATATATGTCTCGCTCCCTTTCG
CTTACAGAAAGGATTTCTTCCGACAGACGTGACCCCAGAAGGCTGCCGAGGGAG
ACTTTAGGACAAGAGGCGTTGTTTTTTTAGGGAAGGAAGAGGAGCCTGTGCAGA
GTTGAACTGGGGTGGAGAGGTGCCTTGTAAAGTATGGTTTCCAGAGATGGTGAG
ACCTCGTAGCTAAGCGTTGTTTCCTCTGCACGTAACTGTGAGTTGTGAACTTGTG
GCTCCTGGGGGAGCGGGACAGGGCCTGGCAGTGCGGGGGAGGGGGCAAGGAAT
TCAGGAAGTTGGCGAGAATTCTTCGTTTTTCATTCTTCGTTGTAAATTGCTTCTAA
ACACTTCTGAATTTTAAATTGCTACCGAGAAGGTCCAGAAACCGTTCTCATCCAA
TTTAGTCACTTTTATTACGTTCCACTTTGCCTCCCCCAATCACTGCGGCGGCTCCC
AGTTTCAGCGTGGCCGCATTTGGAAAAGGCTGAAGGTAGAAAACTCTGGAATGG
AGAGGCTGGCGACTGGAGCAGGATCCAGGGGCTTTAAATTTTAACCTGAATGCG
ATCGAATGCGCGACTCCCAGAAGAGATAGCCAGGCGTCCGCCCGGCCGCTCAAC
CCCCGCCAGGACTCGGAATGCCTTGCTTTTGGTTCTTTTCTACAAGGATGTCTGCG
TGGATATCCCTGGACACTCGATACCCCCCGGGGACCTGCTTTTCCCGCAGCTCCG
CAGACGCGGCGGGAGGGCGCGCTGGCCCCGGCGCTGCTGGGACAGAGGTGAGC
AGCGGGGTGGACATTGACCGATTCGACAACGTTCGGGCTCTAAGGGCTCGGGAA
GGGACCCGGCATTTTACCCTCCAGATTCCCAATCTATTTTTAAAAATAATGTTCA
AAACATATGGGAAAAATAAGTGATTGCTCCTTTTCATGGTCACAGATTGTATTTA
TTTTTTCAGACTGGTTTTCCAACCTAACGAGGGAATGGGTAGTGAATGTTGTTGTT
GTTGTTGTTGTTTATGTTGGTTAATTTCGAAATAAAACTGTTTAACTCGTACAGTT
GATTCAGACAGAAAA
SEQ ID NO: 4:
AACCCCCGCCTAGCCCCTTCACAGCAAATGCGCTTTTTGCCCTGAAGTTGTGTGA
GACCGACCTTAAACTTTCTCCGACCCTATCTCGGGATGCTACGGCTCAGGTCGTG
GTGGTTTCGGGAACCTCAAGACAACATTCCCCAGCTGACCTTTGGGAACTTTAAC
TCGGGTCGTCATGTACGGAAAGGTAGTCTATGGGGTACGCAGAATGGAAAAGGG
CTTCCACATCAGTCGTGTCCGAGGACGCTCTCCAACTTTTGATGTCCCTGGAGAG
TTTCTGATATTTCCCTGGAGAGTTTCAGAAAGGATTTCTTCCGACAGACGTGACC
CCAGAAGGCTGCCGAGGGAGACTTTAGGACAAGAGGCGTTGTTTTTGTGAGAAT
GAATTTTTATACTTTACCCCGACCCCACCCCTACCCCCCGCCCAGGTAAATCCAA
GTAAATGATTTTCTGAAGGTCGGTCTCCTAGAAAGCGTTTAGGCTCTGGTTTTTTG
TTTTGTTTCTATTTTCGTGTTTTCCCAGTTAGGGAAGGAAGAGGAGCCTGTGCAG
AGTTGAACTGGGGTGGAGAGGTGCCTTGTAAAGTATGGTTTCCAGAGATGGTGA
GACCTCGTAGCTAAGCGTTGTTTCCTCTGCACGTAACTGTGAGTTGTGAACTTGT
GGCTCCTGGGGGAGCGGGACAGGGCCTGGCAGTGCGGGGGAGGGGGCAAGGAA
TTCAGGAAGTTGGCGAGAATTCTTCGTTTTTCATTCTTCGTTGTAAATTGCTTCTA
AACACTTCTGAATTTTAAATTGCTACCGAGAAGGTCCAGAAACCGTTCTCATCCA
ATTTAGTCACTTTTATTACGTTCCACTTTGCCTCCCCCAATCACTGCGGCGGCTCC
CAGTTTCAGCGTGGCCGCATTTGGAAAAGGCTGAAGGTAGAAAACTCTGGAATG
GAGAGGCTGGCGACTGGAGCAGGATCCAGGGGCTTTAAATTTTAACCTGAATGC
GATCGAATGCGCGACTCCCAGAAGAGATAGCCAGGCGTCCGCCCGGCCGCTCAA
CCCCCGCCAGGACTCGGAATGCCTTGCTTTTGGTTCTTTTCGTGAGTTCCGACCAA
ATGTTTAAGTGGACTGGGTTTTGCAGAAGCTCATTTGTAAGCACTGACGGAAATG
GGTTGTGGGCATTTGATTTCCTTTGATTCCAACGTTTTGAAATAATGTTTCTCGGA
TTTGCTATTCGAAGCAATTTTCTAAAAACTCATATCTTATTTGCTAAAAATAAACT
CTTTGGCTCAGTTTGTGTCCAGGCTGTGTACGGCCTCCAACCCTGAGCCCCGAGG
AGATCGAGCCCTGGCGCGAGCCTGATCTCTGTCCTGTGTGTTTCTTTGTATTTGCA
AGTACAAGGATGTCTGCGTGGATATCCCTGGACACTCGATACCCCCCGGGGACCT
GCTTTTCCCGCAGCTCCGCAGACGCGGCGGGAGGGCGCGCTGGCCCCGGCGCTG
CTGGGACAGAGGTGAGCAGCGGGGTGGACATTGACCGATTCGACAACGTTCGGG
CTCTAAGGGCTCGGGAAGGGACCCGGCATTTTACCCTCCAGATTCCCAATCTATT
TTTAAAAATAATGTTCAAAACATATGGGAAAAATAAGTGATTGCTCCTTTTCATG
GTCACAGATTGTATTTATTTTTTCAGACTGGTTTTCCAACCTAACGAGGGAATGG
GTAGTGAATGTTGTTGTTGTTGTTGTTGTTTATGTTGGTTAATTTCGAAATAAAAC
TGTTTAACTCGTACAGTTGATTCAGACAG
SEQ ID NO: 5:
AAAGAGACTAGAAGTTGGTCCGCGGTGTCCCGGGAGCCTGGGAGCCGCCTCAGC
CCGCCCGGAGGGAAGGAGGGAAACACCGTCGGAGCCGACCGCGGCCTGGAGAG
GGCCGGAGAGCCGGAGGCCAGGGGCCCGCGACGAGTAGGGGTGGAGGGGAGGA
AAGCGGGGAGATCGCGCGAGGAGGAAGCAGCCGAACCTCGGGCGGGCGCTGCG
CATGGAGAACGGCCGCGGCGGAGGGTGCTCCCAGGCGCGGAGAGAAGCCGAGG
GCGCCAGCGGGCGAGCGCGAGTCCGGGGTCTGCGCCGCGCTGCTGGTGAATAAA
AAGGAGAGAGGAGGCGCCTCTTACTGCTCTGCCGGAAAACTGCAGCCTGGGCTC
CTGATTGGACTCACCGAGCCCTAAACAAACAGCGCTCGCCGAAGGGTGCCAGGC
TGTGGGTCGGAACTGCGCTCAGCGAGCAGCTGGGCGCTGGAGTTGTGTGAGACC
GACCTTAAACTTTCTCCGACCCTATCTCGGGATGCTACGGCTCAGGTCGTGGTGG
TTTCGGGAACCTCAAGACAACATTCCCCAGCTGACCTTTGGGAACTTTAACTCGG
GTCGTCATGTACGGAAAGGTAGTCTATGGGGTACGCAGAATGGAAAAGGGCTTC
CACATCAGTCGTGTCCGAGGACGCTCTCCAACTTTTGATGTCCCTGGAGAGTTTC
TGATATTTCCCTGGAGAGTTTCAGTTTCAGCGTGGCCGCATTTGGAAAAGGCTGA
AGGTAGAAAACTCTGGAATGGAGAGGCTGGCGACTGGAG
SEQ ID NO: 6:
AAAGGCTGAAGGTAGAAAACTCTGGAATGGAGAGGCTGGCGACTGGAGCAGGA
TCCAGGGGCTTTAAATTTTAACCTGAATGCGATCGAATGCGCGACTCCCAGAAGA
GATAGCCAGGCGTCCGCCCGGCCGCTCAACCCCCGCCAGGACTCGGAATGCCTT
GCTTTTGGTTCTTTTCCCTGTTATCTAGGCCACCCAATGACCGACGAAAGATTTTC
CCTCCCTGCTTCTCCCGTGGCCGCTTTAAACCAAATAAATTTAGCTATTAAGGGC
CAAGATGTCTGACAAAAGATCGAAAGGTGTCTTTCCGTGATCGCTTAAAGAAGC
GAAGGAGAAGCTTGCGGCTCTCAAATCCCGATAAAGAAATTCCGCGCCGGCGCC
GGCCTCCCTCCCCGCGGCCTGGTTGCTCCCTGACGCCCTGAGACCCCGACTTCGG
CGGCCGCAGACCTCGGACGGGCCCGGCGGGACCTGTGGCTGCCGCCTGGGGAAG
GTGGAGGTCCCCGGGCTGCGGGGCCAGGGAGTGCCAGGCCCTAGGAGACGGGC
GGACAAGGGGGTCCCCGGGAGCTCTGGAGCTCCAGGTGGGAGCTGGGTATCCTA
AAACGGCCCCCGTGCACTTCGCGGAAAAACTAAGCCAAGCTCAGGGGGCCGAAA
TTACCACTCCGTTTTGATTATTTTCTGATACTAAATAATAAAGATAAAACCAAAC
TGCC
SEQ ID NO: 7:
ACGTCAGGTGGTGGCGGCGGAAACTTGCAGGTCGCCCCCACCCCCAGCGCCGGC
TCCCGCCGGCGGCCACTTCGGGGAAGGACCGCGCGGAGCCAGCGCCGGGGTCCC
CGGGCGGTCGGAATCGAGGCCCGGCGCGTTGCGCAGGCCAGGCCCGGCCCGGAG
GCCCCTTGCCCGGCGCAGCCCCCGGCCGACCCCCGTGCGCCGCCGGCGGCCGCC
AGCACGGCGCGGAGGTGGGCTAGCGGCGGCGGCGCACGAAAGGTCACCGCGGT
GGCATTCCTCTCCGCGCTGTTTACGTGACTTAATGTACTCGCTGCGCCGCGGCCT
GTTATCTAGGCCACCCAATGACCGACGAAAGATTTTCCCTCCCTGCTTCTCCCGT
GGCCGCTTTAAACCAAATAAATTTAGCTATTAAGGGCCAA
SEQ ID NO: 8:
AGTTATCACAACTCTGTACAAACTGATACGGTCCCTAGCGGGCAAAAGGGACTG
GAAATGAACGTCGCGGGGTTATCAGCGCCGATCTGTCAGCGGCAACTCGCGCGG
CCGCAGGCTCGTCCGCTGCAGGAGCCGTGATATATGTCTCGCTCCCTTTCGCTTA
CAGAAAGGATTTCTTCCGACAGACGTGACCCCAGAAGGCTGCCGAGGGAGACTT
TAGGACAAGAGGCGTTGTTTTTGTGAGAATGAATTTTTATACTTTACCCCGACCC
CACCCCCTACCCCCCGCCCAGGTAAATCCAAGTAAATGATTTTCTGAAGGTCGGT
CTCCTAGAAAGCGTTTAGGCTCTGGTTTTTTGTTTTGTTTCTATTTTCGTGTTTTCC
CAGTTAGGGAAGGAAGAGGAGCCTGTGCAGAGTTGAACTGGGGTGGAGAGGTG
CCTTGTAAAGTATGGTTTCCAGAGATGGTGAGACCTCGTAGCTAAGCGTTGTTTC
CTCTGCACGTAACTGTGAGTTGTGAACTTGTGGCTCCTGGGGGAGCGGGACAGG
GCCTGGCAGTGCGGGGGAGGGGGCAAGGAATTCAGGAAGTTGGCGAGAATTCTT
CGTTTTTCATTCTTCGTTGTAAATTGCTTCTAAACACTTCTGAATTTTAAATTGCTA
CCGAGAAGGTCCAGAAACCGTTCTCATCCAATTTAGTCACTTTTATTACGTTCCA
CTTTGCCTCCCCCAATCACTGCGGCGGCTCCCAGTTTCAGCGTGGCCGCATTTGG
AAAAGGCTGAAGGTAGAAAACTCTGGAATGGAGAGGCTGGCGACTGGAGCAGG
ATCCAGGGGCTTTAAATTTTAACCTGAATGCGATCGAATGCGCGACTCCCAGAAG
AGATAGCCAGGCGTCCGCCCGGCCGCTCAACCCCCGCCAGGACTCGGAATGCCT
TGCTTTTGGTTCTTTTCGTGAGTTCCGACCAAATGTTTAAGTGGACTGGGTTTTGC
AGAAGCTCATTTGTAAGCACTGACGGAAATGGGTTGTGGGCATTTGATTTCCTTT
GATTCCAACGTTTTGAAATAATGTTTCTCGGATTTGCTATTCGAAGCAATTTTCTA
AAAACTCATATCTTATTTGCTAAAAATAAACTCTTTGGCTCAGTTTGTGTCCAGGC
TGTGTACGGCCTCCAACCCTGAGCCCCGAGGAGATCGAGCCCTGGCGCGAGCCT
GATCTCTGTCCTGTGTGTTTCTTTGTATTTGCAAGTACAAGGATGTCTGCGTGGAT
ATCCCTGGACACTCGATACCCCCCGGGGACCTGCTTTTCCCGCAGCTCCGCAGAC
GCGGCGGGAGGGCGCGCTGGCCCCGGCGCTGCTGGGACAGAGGTGAGCAGCGG
GGTGGACATTGACCGATTCGACAACGTTCGGGCTCTAAGGGCTCGGGAAGGGAC
CCGGCATTTTACCCTCCAGATTCCCAATCTATTTTTAAAAATAATGTTCAAAACAT
ATGGGAAAAATAAGTGATTGCTCCTTTTCATGGTCACAGATTGTATTTATTTTTTC
AGACTGGTTTTCCAACCTAACGAGGGAATGGGTAGTGAATGTTGTTGTTGTTGTT
GTTGTTTATGTTGGTTAATTTCGAAATAAAACTGTTTAACTCGTACAGTTGATTCA
GACAGAAAACCA
SEQ ID NO: 9:
GATCTGTCAGCGGCAACTCGCGCGGCCGCAGGCTCGTCCGCTGCAGGAGCCGTG
ATATATGTCTCGCTCCCTTTCGCTTACAGAAAGGATTTCTTCCGACAGACGTGAC
CCCAGAAGGCTGCCGAGGGAGACTTTAGGACAAGAGGCGTTGTTTTTTTAGGGA
AGGAAGAGGAGCCTGTGCAGAGTTGAACTGGGGTGGAGAGGTGCCTTGTAAAGT
ATGGTTTCCAGAGATGTTTCAGCGTGGCCGCATTTGGAAAAGGCTGAAGGTAGA
AAACTCTGGAATGGAGAGGCTGGCGACTGGAGCAGGATCCAGGGGCTTTAAATT
TTAACCTGAATGCGATCGAATGCGCGACTCCCAGAAGAGATAGCCAGGCGTCCG
CCCGGCCGCTCAACCCCCGCCAGGACTCGGAATGCCTTGCTTTTGGTTCTTTTCTA
CAAGGATGTCTGCGTGGATATCCCTGGACACTCGATACCCCCCGGGGACCTGCTT
TTCCCGCAGCTCCGCAGACGCGGCGGGAGGGCGCGCTGGCCCCGGCGCTGCTGG
GACAGAGGTGAGCAGCGGGGTGGACATTGACCGATTCGACAACGTTCGGGCTCT
AAGGGCTCGGGAAGGGACCCGGCATTTTACCCTCCAGATTCCCAATCTATTTTTA
AAAATAATGTTCAAAACATATGGGAAAAATAAGTGATTGCTCCTTTTCATGGTCA
CAGATTGTATTTATTTTTTCAGACTGGTTTTCCAACCTAACGAGGGAATGGGTAG
TGAATGTTGTTGTTGTTGTTGTTGTTTATGTTGGTTAATTTCGAAATAAAACTGTT
TAACTCGTACAGTTGATTCAGACAGAAAA
SEQ ID NO: 10:
AGAAAGGATTTCTTCCGACAGACGTGACCCCAGAAGGCTGCCGAGGGAGACTTT
AGGACAAGAGGCGTTGTTTTTGGAAGGAAGAGGAGCCTGTGCAGAGTTGAACTG
GGGTGGAGAGGTGCCTTGTAAAGTATGGTTTCCAGAGATGGTGAGACCTCGTAG
CTAAGCGTTGTTTCCTCTGCACGTAACTTTTCAGCGTGGCCGCATTTGGAAAAGG
CTGAAGGTAGAAAACTCTGGAATGGAGAGGCTGGCGACTGGAGCAGGATCCAG
GGGCTTTAAATTTTAACCTGAATGCGATCGAATGCGCGACTCCCAGAAGAGATA
GCCAGGCGTCCGCCCGGCCGCTCAACCCCCGCCAGGACTCGGAATGCCTTGCTTT
TGGTTCTTTTCTACAAGGATGTCTGCGTGGATATCCCTGGACACTCGATACCCCC
CGGGGACCTGCTTTTCCCGCAGCTCCGCAGACGCGGCGGGAGGGCGCGCTGGCC
CCGGCGCTGCTGGGACAGAGGTGAGCAGCGGGGTGGACATTGACCGATTCGACA
ACGTTCGGGCTCTAAGGGCTCGGGAAGGGACCCGGCATTTTACCCTCCAGATTCC
CAATCTATTTTTAAAAATAATGTTCAAAACATATGGGAAAAATAAGTGATTGCTC
CTTTTCATGGTCACAGATTGTATTTATTTTTTCAGACTGGTTTTCCAACCTAACGA
GGGAATGGGTAGTGAATGTTGTTGTTGTTGTTGTTGTTTATGTTGGTTAATTTCGA
AATAAAACTGTTTAACTCGTA
SEQ ID NO: 11:
GATATATGTCTCGCTCCCTTTCGCTTACAGAAAGGATTTCTTCCGACAGACGTGA
CCCCAGAAGGCTGCCGAGGGAGACTTTAGGACAAGAGGCGTTGTTTTTTTTCAGC
GTGGCCGCATTTGGAAAAGGCTGAAGGTAGAAAACTCTGGAATGGAGAGGCTGG
CGACTGGAGCAGGATCCAGGGGCTTTAAATTTTAACCTGAATGCGATCGAATGC
GCGACTCCCAGAAGAGATAGCCAGGCGTCCGCCCGGCCGCTCAACCCCCGCCAG
GACTCGGAATGCCTTGCTTTTGGTTCTTTTCTACAAGGATGTCTGCGTGGATATCC
CTGGACACTCGATACCCCCCGGGGACCTGCTTTTCCCGCAGCTCCGCAGACGCGG
CGGGAGGGCGCGCTGGCCCCGGCGCTGCTGGGACAGAGGTGAGCAGCGGGGTG
GACATTGACCGATTCGACAACGTTCGGGCTCTAAGGGCTCGGGAAGGGACCCGG
CATTTTACCCTCCAGATTCCCAATCTATTTTTAAAAATAATGTTCAAAACATATGG
GAAAAATAAGTGATTGCTCCTTTTCATGGTCACAGATTGTATTTATTTTTTCAGAC
TGGTTTTCCAACCTAACGAGGGAATGGGTAGTGAATGTTGTTGTTGTTGTTGTTGT
TTATGTTGGTTAATTTCGAAATAAAACTGTTTAACTCGTA
SEQ ID NO: 12:
AGTTATCACAACTCTGTACAAACTGATACGGTCCCTAGCGGGCAAAAGGGACTG
GAAATGAACGTCGCGGGGTTATCAGCGCCGATCTGTCAGCGGCAACTCGCGCGG
CCGCAGGCTCGTCCGCTGCAGGAGCCGTGATATATGTCTCGCTCCCTTTCGCTTA
CAGAAAGGATTTCTTCCGACAGACGTGACCCCAGAAGGCTGCCGAGGGAGACTT
TAGGACAAGAGGCGTTGTTTTTGTGAGAATGAATTTTTATACTTTACCCCGACCC
CACCCCCTACCCCCCGCCCAGGTAAATCCAAGTAAATGATTTTCTGAAGGTCGGT
CTCCTAGAAAGCGTTTAGGCTCTGGTTTTTTGTTTTGTTTCTATTTTCGTGTTTTCC
CAGTTAGGGAAGGAAGAGGAGCCTGTGCAGAGTTGAACTGGGGTGGAGAGGTG
CCTTGTAAAGTATGGTTTCCAGAGATGGTGAGACCTCGTAGCTAAGCGTTGTTTC
CTCTGCACGTAACTGTGAGTTGTGAACTTGTGGCTCCTGGGGGAGCGGGACAGG
GCCTGGCAGTGCGGGGGAGGGGGCAAGGAATTCAGGAAGTTGGCGAGAATTCTT
CGTTTTTCATTCTTCGTTGTAAATTGCTTCTAAACACTTCTGAATTTTAAATTGCTA
CCGAGAAGGTCCAGAAACCGTTCTCATCCAATTTAGTCACTTTTATTACGTTCCA
CTTTGCCTCCCCCAATCACTGCGGCGGCTCCCAGTTTCAGCGTGGCCGCATTTGG
AAAAGGCTGAAGGTAGAAAACTCTGGAATGGAGAGGCTGGCGACTGGAGCAGG
ATCCAGGGGCTTTAAATTTTAACCTGAATGCGATCGAATGCGCGACTCCCAGAAG
AGATAGCCAGGCGTCCGCCCGGCCGCTCAACCCCCGCCAGGACTCGGAATGCCT
TGCTTTTGGTTCTTTTCTACAAGGATGTCTGCGTGGATATCCCTGGACACTCGATA
CCCCCCGGGGACCTGCTTTTCCCGCAGCTCCGCAGACGCGGCGGGAGGGCGCGC
TGGCCCCGGCGCTGCTGGGACAGAGGTGAGCAGCGGGGTGGACATTGACCGATT
CGACAACGTTCGGGCTCTAAGGGCTCGGGAAGGGACCCGGCATTTTACCCTCCA
GATTCCCAATCTATTTTTAAAAATAATGTTCAAAACATATGGGAAAAATAAGTGA
TTGCTCCTTTTCATGGTCACAGATTGTATTTATTTTTTCAGACTGGTTTTCCAACCT
AACGAGGGAATGGGTAGTGAATGTTGTTGTTGTTGTTGTTGTTTATGTTGGTTAAT
TTCGAAATAAAACTGTTTAACTCGTA
SEQ ID NO: 13:
GAAAGGATTTCTTCCGACAGACGTGACCCCAGAAGGCTGCCGAGGGAGACTTTA
GGACAAGAGGCGTTGTTTTTTTAGGGAAGGAAGAGGAGCCTGTGCAGAGTTGAA
CTGGGGTGGAGAGGTGCCTTGTAAAGTATGGTTTCCAGAGATGGTGAGACCTCGT
AGCTAAGCGTTGTTTCCTCTGCACGTAACTTTTCAGCGTGGCCGCATTTGGAAAA
GGCTGAAGGTAGAAAACTCTGGAATGGAGAGGCTGGCGACTGGAGCAGGATCC
AGGGGCTTTAAATTTTAACCTGAATGCGATCGAATGCGCGACTCCCAGAAGAGA
TAGCCAGGCGTCCGCCCGGCCGCTCAACCCCCGCCAGGACTCGGAATGCCTTGCT
TTTGGTTCTTTTCGTGAGTTCCGACCAAATGTTTAAGTGGACTGGGTTTTGCAGAA
GCTCATTTGTAAGCACTGACGGAAATGGGTTGTGGGCATTTGATTTCCTTTGATTC
CAACGTTTTGAAATAATGTTTCTCGGATTTGCTATTCGAAGCAATTTTCTAAAAAC
TCATATCTTATTTGCTAAAAATAAACTCTTTGGCTCAGTTTGTGTCCAGGCTGTGT
ACGGCCTCCAACCCTGAGCCCCGAGGAGATCGA
SEQ ID NO: 14:
GTCCGCTGCAGGAGCCGTGATATATGTCTCGCTCCCTTTCGCTTACAGAAAGGAT
TTCTTCCGACAGACGTGACCCCAGAAGGCTGCCGAGGGAGACTTTAGGACAAGA
GGCGTTGTTTTTTTAGGGAAGGAAGAGGAGCCTGTGCAGAGTTGAACTGGGGTG
GAGAGGTGCCTTGTAAAGTATGGTTTCCAGAGATGGTGAGACCTCGTAGCTAAG
CGTTGTTTCCTCTGCACGTAACTGTGAGTTGTGAACTTGTGGCTCCTGGGGGAGC
GGGACAGGGCCTGGCAGTGCGGGGGAGGGGGCAAGGAATTCAGGAAGTTGGCG
AGAATTCTTCGTTTTTCATTCTTCGTTGTAAATTGCTTCTAAACACTTCTGAATTTT
AAATTGCTACCGAGAAGGTCCAGAAACCGTTCTCATCCAATTTAGTCACTTTTAT
TACGTTCCACTTTGCCTCCCCCAATCACTGCGGCGGCTCCCAGTTTCAGCGTGGC
CGCATTTGGAAAAGGCTGAAGGTAGAAAACTCTGGAATGGAGAGGCTGGCGACT
GGAGCAGGATCCAGGGGCTTTAAATTTTAACCTGAATGCGATCGAATGCGCGAC
TCCCAGAAGAGATAGCCAGGCGTCCGCCCGGCCGCTCAACCCCCGCCAGGACTC
GGAATGCCTTGCTTTTGGTTCTTTTCGTGAGTTCCGACCAAATGTTTAAGTGGACT
GGGTTTTGCAGAAGCTCATTTGTAAGCACTGACGGAAATGGGTTGTGGGCATTTG
ATTTCCTTTGATTCCAACGTTTTGAAATAATGTTTCTCGGATTTGCTATTCGAAGC
AATTTTCTAAAAACTCATATCTTATTTGCTAAAAATAAACTCTTTGGCTCA
SEQ ID NO: 15:
GCTGTGGGTCGGAACTGCGCTCAGCGAGCAGCTGGGCGCTGGAGGTAAATCCAA
GTAAATGATTTTCTGAAG

It is noted that SEQ ID NO: 15 is a relatively short sequence and does not comply with the traditional definition of a long non-coding RNA. In some embodiments, provided herein are synthetic GATA6-AS1 lncRNA (e.g., SEQ ID NO: 1 or sequences with at least 60%, 65%, 70%, 75%, 80%, 85%, 90%, 91%, 92%, 93%, 94%, 95%, 96%, 97%, 98%, 99%, or higher sequence identity with SEQ ID NO:1).

As used herein, the term “identity” refers to two or more nucleic acid sequences or subsequences that are the same or have a specified percentage of nucleotides that are the same, e.g., about 60%, 65%, 70%, 75%, 80%, 85%, 90%, 91%, 92%, 93%, 94%, 95%, 96%, 97%, 98%, 99%, or higher identity over a specified region. As known to a person of skill in the art, to examine identity between two or more sequences the sequences should be aligned.

As used herein, “pharmaceutical composition” refers to the combination of an active agent, compound, or ingredient (e.g., the GATA6-AS1 lncRNA of the invention) with a pharmaceutically acceptable carrier or excipient, making the composition suitable for diagnostic, therapeutic or preventive use.

As used herein the term “pharmaceutically acceptable carrier, excipient or diluent” includes all solvents, dispersion media, coatings, antibacterial and antifungal agents, and the like, as known in the art. The carrier can be solvent or dispersion medium containing, for example, water, ethanol, polyol (for example, glycerol, propylene glycol, and liquid polyethylene glycol, and the like), suitable mixtures thereof, and vegetable oils. Each carrier should be both pharmaceutically and physiologically acceptable in the sense of being compatible with the other ingredients and not injurious to the subject. Except as any conventional media or agent is incompatible with the active ingredient, its use in the therapeutic composition is contemplated.

Administration according to the present invention may be performed by any one of the following routes: intra-rectal administration (also referred to as rectal administration), oral administration, parenteral, intravenous, intramuscular, intraperitoneal, intrathecal, transdermal, transcutaneous, or subcutaneous injection.

In certain embodiments the composition of the present disclosure is delivered to the gastrointestinal mucosa, e.g., by intra-rectal administration.

The GATA6-AS1 lncRNA of the invention can be ferried into cells using viral vector based or non-viral delivery systems. Non-viral delivery systems include for example, polymer-based, lipid-based, and conjugate-based drug delivery systems.

Methods of RNA delivery are known in the art, see for example Paunovska et al Nature Reviews Genetics 23, 265-280 (2022).

In some embodiments, the GATA6-AS1 lncRNA of the invention is delivered to the individual using lipids or lipid-based nanoparticles. Based on the size of the hydrophilic head group relative to the size of the hydrophobic tail or tails, lipids form distinct structures including micelles, liposomes, and lipid nanoparticles (LNPs). Thus, in one embodiment the present invention concerns lipid nanoparticles comprising the GATA6-AS1 lncRNA of the invention. LNPs typically include variations of the following basic components: a cationic or ionizable lipid, cholesterol, a helper lipid, and a poly (ethylene glycol) (PEG)-lipid. Such lipid-based delivery systems may be complexed with nucleic acids. Lipids may be synthesized using Michael addition-based, epoxide-based, and alcohol-based reactions. GATA6-AS1 lncRNA LNP formulations can be prepared for example using the NanoAssemblr microfluidic mixing system, in which mRNA molecules self-assemble with ionizable lipids under acidic conditions to form highly uniform nanoparticles. Non-limiting examples of lipids that may be used to deliver the GATA6-AS1 lncRNA of the invention include C12-200 synthesized using epoxide-amine chemistry; cKK-E12, a peptide-like lipid compound; DLin-KC2-DMA, an ionizable lipid; and DLin-MC 3-DMA.

In some embodiments, the GATA6-AS1 lncRNA of the invention is delivered to the individual using polymers or polymer-based nanoparticles.

Non-limiting examples include modified poly (lactic-co-glycolic acid) (PLGA) containing an added cationic chemical group such as chitosan, modified polyethylenimine (PEI) and poly(l-lysine) (PLL), e.g., nanoparticles made with PEG-grafted PEI, and cyclodextrin-PEI conjugates, iron oxide nanoparticles that are optionally surface-modified with PLL, poly (beta-amino ester)s (PBAEs), which are synthesized by conjugating amine monomers to diacrylates, lipid-polymer hybrids and dendrimers that are synthesized with cationic groups, such as poly(amidoaraine) (PAMAM) or PLL.

The various delivery systems may target specific cell types or body tissues either passively or actively. Accordingly, in some embodiments, the GATA6-AS1 lncRNA of the invention is delivered to the individual using active targeting. Namely, a ligand that binds a specific biomolecule is added to the delivery system. For example, the nanoparticles can be tagged with antibodies against cell surface antigens of the target tissue or be tagged using any entity capable of directing the selected delivery system (e.g., the nanoparticles) to the gastrointestinal mucosa.

In one embodiment, the present invention provides epithelial cells targeted LNPs.

In specific embodiments the composition as herein defined is administered in a single-dose regimen. In further specific embodiments the composition as herein defined is administered in a multiple-dose regimen.

The present disclosure further provides a kit comprising the composition of the invention and instructions for the use of the composition.

The effective amount of the composition according to the invention for purposes herein defined is determined by such considerations as are known in the art to treat chronic intestinal inflammation and/or inflammatory bowel disease (IBD). For any preparation used in the methods of the invention, the dosage can be estimated initially from in vitro cell culture assays or based on suitable animal models.

In another aspect, the present inventio provides the use of GATA6-AS1 lncRNA as a biomarker for chronic intestinal inflammation and/or IBD.

The invention also provides an in vitro method for chronic intestinal inflammation and/or IBD diagnosis of an individual, wherein the method comprises the step of determining the amount of GATA6-AS1 lncRNA in a biological sample from said individual, wherein a reduced amount of GATA6-AS1 lncRNA is indicative of chronic intestinal inflammation and/or IBD.

In one embodiment, prior to treatment with the GATA6-AS1 lncRNA, the individual has been diagnosed by a method that comprises measuring GATA6-AS1 lncRNA in the individual, detecting a reduced level of GATA6-AS1 lncRNA as compared with a reference level, and thereby providing a positive diagnosis for chronic intestinal inflammation and/or IBD in the individual.

In another embodiment, the administration of the GATA6-AS1 lncRNA or functional fragment thereof to the individual is following diagnosis of chronic intestinal inflammation and/or IBD in the individual using a method that comprises measuring GATA6-AS1 lncRNA in the individual, detecting reduced level of GATA6-AS1 lncRNA as compared with a reference level, and thereby providing a positive diagnosis for chronic intestinal inflammation and/or IBD in the individual.

As used herein, the term “diagnosis” or “diagnosed” refers to the determination as to whether an individual is likely to be affected by chronic intestinal inflammation and/or IBD. The skilled artisan often makes a diagnosis based on one or more diagnosis markers or biomarkers, the presence, absence, or amount of which is indicative of the presence or absence of chronic intestinal inflammation and/or IBD. The term “diagnosis” also encompasses providing information useful for diagnosis.

As used herein the term “biomarker” refers to a biological parameter that aids the diagnosis of chronic intestinal inflammation and/or IBD. It is a measurable indicator of the presence of this disease. In the context of the present invention, this term refers particularly to GATA6-AS1 lncRNA. GATA6-AS1 lncRNA is produced by normal epithelia of the gut; however, it is produced by much lower levels under disease conditions. GATA6-AS1 lncRNA can be found in the gut epithelial cells.

The terms “level” and “amount” are used interchangeably herein and may refer to an absolute quantification of a molecule in a sample, or to a relative quantification of a molecule in a sample, i.e., relative to another value such as relative to a reference value, or to a range of values. These values or ranges may be obtained from a single individual or from a group of patients, e.g., the general population, patient cohorts, and the like.

The terms “biological sample” and “sample” are used interchangeably herein to denote any sample containing mRNA derived from the gastrointestinal tract of the individual. Examples of such samples include biopsies, organs, tissues, or cell samples, in particular duodenum, ileum, and rectum samples. Preferably, the biological sample is a biopsy, optionally taken during colonoscopy.

GATA6-AS1 lncRNA levels in a patient's gastrointestinal system can be measured using any method known in the art, for example by obtaining an epithelial or mucosal sample (e.g., a biopsy) from the patient's gastrointestinal system and performing a transcriptome analysis, i.e., obtaining mRNA sequence data from said sample.

Several methods and kits for extracting nucleic acids, e.g., mRNA, contained in the sample are available for the person skilled in the art. For example, extraction may rely on lytic enzymes or chemical solutions or can be done with nucleic-acid-binding resins following the manufacturer's instructions.

In one embodiment, the cells and cell fragments present in the sample, preferably the gut biopsy, are collected, preferably by centrifugation. A total nucleic acid extraction is then carried out. Non-limiting examples are phenol/chloroform or Trizol extraction methods. Total nucleic acid extraction may also be carried out using a solid phase band method on silica beads. Numerous nucleic acid extraction methods exist, and thus other methods can be used in accordance with the present invention.

After total nucleic acid extraction, DNA may be degraded to conserve only the RNA molecules, for example using a deoxyribonuclease enzyme.

A variety of nucleic acids quantification techniques, well known by the skilled person, can be used to determine the amount of GATA6-AS1 lncRNA in the sample, or in the RNAs extracted from said sample. These techniques can be adapted to the type of sample, the sensitivity of the quantification desired, the amount of nucleic acid in the sample, and the like.

Measurement of the amount of GATA6-AS1 lncRNA can be direct or indirect. Direct quantification may be performed by hybridization, e.g., with a labeled specific probe, for example by hybridization of a fluorescent labeled specific probe immobilized directly or indirectly on a solid support, e.g., by the Nanostring method.

Alternatively, the amount of GATA6-AS1 lncRNA can be determined indirectly, especially after its conversion to cDNA, preferably by amplification, especially quantitative amplification, e.g., an amplification method coupled to quantitative real-time PCR (RT-PCR).

Other methods of amplification include ligase chain reaction (LCR), transcription-mediated amplification (TMA), strand displacement amplification (SDA), nucleic acid sequence-based amplification (NASBA), and microarray analysis. These methods are well known by the skilled person.

In one embodiment, and as shown in the Examples below, RNA is isolated from biopsies obtained during diagnostic colonoscopy using the Qiagen AllPrep RNA/DNA Mini Kit. PolyA-RNA selection, fragmentation, cDNA synthesis, adaptor ligation, TruSeq RNA sample library preparation (Illumina, San Diego, CA), and paired-end 75 bp sequencing is performed.

GATA6-AS1 lncRNA expression can be calculated from the transcriptomics raw data using known software, for example, as described in the Examples below.

The term “about” as used herein indicates values that may deviate up to 1%, more specifically, 5%, more specifically 10%, more specifically 15%, and in some cases up to 20% higher or lower than the value referred to, the deviation range including integer values, and, if applicable, non-integer values as well, constituting a continuous range. Disclosed and described, it is to be understood that this invention is not limited to the specific examples, methods steps, and compositions disclosed herein as such methods steps and compositions may vary somewhat. It is also to be understood that the terminology used herein is used for the purpose of describing specific embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

### EXAMPLES

### Methods

Cohorts

PROTECT cohort (Test cohort 1) was described previously [9,10]. PROTECT is a multicenter inception cohort study including treatment naïve children aged 4-17 years with a diagnosis of UC. Disease extent, clinical activity at diagnosis (PUCAI range 0-85), Mayo endoscopic scope (grade 1-3), and total Mayo score (range 0-12) were captured. PUCAI less than 10 denoted inactive disease or remission, 10-30 denoted mild disease, 35-60 denoted moderate disease, and 65 or higher denoted severe disease. A central pathologist blinded to clinical data examined a single rectal biopsy. Depending on initial PUCAI score, patients received initial treatment with either mesalamine (mild disease), or corticosteroids (moderate and severe disease), with some physician discretion allowed. Week 4 (W4) remission outcome defined as PUCAI <10 without additional therapy or colectomy, Week 52 as CS-free remission (SFR) with no therapy beyond mesalazine, and occurrence of colectomy within 3 years were recorded. Rectal mucosal biopsies from a representative sub-cohort of 206 PROTECT UC patients and 20 age and gender matched non-IBD controls underwent high coverage transcriptomic profiling using Illumina RNAseq. The 20 controls at Cincinnati Children's Hospital Medical Center were included in the current analyses as non-IBD controls after clinical endoscopic, and biopsies evaluation demonstrated no histologic and endoscopic inflammation.

SOURCE cohort (Test cohort 2) included 8 treatment naïve Crohn disease and 12 non-IBD controls from the Sheba Medical Center. Age, gender, endoscopic findings, diagnostic calprotectin, and CRP were recorded. Sheba Institutional Review Board approved the protocol and safety monitoring plan. Informed consent was obtained for each participant.

SEEM celiac cohort (Test cohort 3) was described previously and included 17 celiac subjects and 25 controls from Cincinnati Children's Hospital Medical Center (CCHMC). Controls were subjects who were investigated for various gastrointestinal symptoms including abdominal pain but had normal endoscopic and histologic findings. Celiac disease diagnosis was based on previously described algorithms including positive IgA autoantibodies against tissue transglutaminase (anti-TTG) and histologic features.

Validation cohorts included the RISK cohort, a treatment naïve pediatric IBD cohort that was used to validate the baseline CD ileal dataset and the UC rectal gene expression dataset [9,15] and the previously published celiac cohort [14].

Epithelial and single cell datasets. Isolated epithelial dataset included reanalyzes of a publicly available adult human colon UC single cell data set, which contains 366,650 cells from 18 UC and 12 healthy adult colons, to examine cell-specific trends in gene expression [12].

RNA Extraction and RNA-Seq Analysis

All transcriptomics analyses started from the raw FATSQ files using a similar pipeline. PROTECT (GSE109142), SOURCE (GSE199906), and SEEM (GSE159495) test cohorts were processed and sequenced using the same laboratory pipeline, sequencing library at the Gene and Protein Expression and Bioinformatics cores of the National Institutes of Health (NIH)-supported Cincinnati Children's Hospital Research Foundation Digestive Health Center. RNA was isolated from biopsies obtained during diagnostic colonoscopy using the Qiagen AllPrep RNA/DNA Mini Kit. PolyA-RNA selection, fragmentation, cDNA synthesis, adaptor ligation, TruSeq RNA sample library preparation (Illumina, San Diego, CA), and paired-end 75 bp sequencing was performed. Median read depth in PROTECT was ˜43M (37-52M IQR), in SOURCE 39M (31-46M IQR, and ˜34M (33-46M IQR) in SEEM. RISK treatment naïve pediatric patients' rectal biopsies and ileal biopsies (rectal GSE117993, ileal GSE101794) were used as validation cohorts for UC and CD respectively using single end 75 bp mRNA sequencing [9, 15]. Those biopsies had similar RNA extractions at Cincinnati Children's and were also sequenced at the (NIH)-supported Cincinnati Children's Hospital Research Foundation Digestive Health Center. Another celiac cohort (PRJNA528755[14]) was used for validation, this cohort used KAPA stranded mRNA-Seq.

Reads were quantified by kallisto [19] version 42.5 using Gencode v24 as the reference genome. Kallisto output files were summarized to gene level using R package tximport version 1.12.3 [20]. mRNA genes with Transcripts per Million (TPM) values above 1 in at least 20% of the samples were used in downstream analysis. Differentially expressed genes between cases and controls, or mild and severe UC had fold change (FC)≥1.5 and false discovery rate (FDR) ≤0.05 using R package DESeq2 version 1.30.1.

Principal Coordinates Analysis (PCA) was performed to summarize variation in gene expression between patients, and principal components (PC) values were extracted for downstream analyses. Clinical features data were correlated to PC values using Spearman's correlation. Unsupervised hierarchical clustering using Euclidean distance metric and Ward's linkage rule was used to test for groups of rectal biopsies with similar patterns of gene expression.

Identifying Co-Expressed Gene Modules

Weighted gene co-expression network analysis (WGCNA) was implemented to identify modules of co-expressed genes, using R WGCNA package version 1.69-81. WGCNA identifies co-expressed gene clusters using pairwise correlations between gene expression profiles. A signed version of WGCNA was used to distinguish between positively and negatively correlated genes. Gene co-expression similarities are converted to signed adjacency values using the power adjacency function, with a β parameter of 5 for lncRNA WGCNA analysis, and a β parameter of 12 for lncRNA and protein-coding genes analysis. The topological similarities represent the interconnectedness of two genes in a gene co-expression network. Average linkage hierarchical clustering is implemented on TOM-based dissimilarities to detect modules of strongly correlated genes across all samples. The cluster sensitivity parameter (deepSplit) was set to its default value of 2 to identify balanced genes modules while the minimum number of genes in a module (minModuleSize) is set to 30 genes. maxBlockSize is set to 20000 to include all genes in one block. For each module, the first principal component referred to as the eigengene, is considered the module representative. A module summarizes the expression levels of all the genes in a module. Candidate modules are identified based on the strength and significance (Student's asymptotic p-value) of the respective module eigengenes with the phenotypic traits including the disease status. The focus was on modules significantly associated with disease status, with p<0.001. The module membership (MM) score signifies the importance of a gene (connectivity-based) within the module and is calculated as the Pearson correlation coefficient between a gene's expression profile and the module eigengene.

This analysis was performed separately for lncRNA only, and for lncRNA and protein coding genes.

ToppGene and ToppCluster software were used to perform Gene Set Enrichment Analyses (GSEA) of the protein coding genes within the modules in the lncRNA and protein coding genes WGCNA analysis, and visualization of the networks was obtained using Cytoscape.v3.0.2.

Random Forest ROC

The random forest analysis was performed in R package randomForest version 4.6-14 with default parameters. For each disease and location, random forest was trained on control and disease samples from the main cohort and tested on the validation cohort. Main cohort expressed genes were used, and main samples were randomly chosen to create equal sized disease and control groups. The randomForest package out-of-box estimate of error rate was used to estimate the main cohorts' classification, and the predict function for the validation cohorts. R AUC package version 0.3.0 was used to calculate the Receiver operating characteristic (ROC) curve. Analysis was performed separately for lncRNAs and protein-coding genes.

Similar and Unique Genes Between Diseases and Locations

Identifying a validated specific disease signal for each disease and location: For each of the 3 main cohorts (PROTECT, SOURCE, SEEM) and the validation cohorts (RISK, Leonard et al. 15 [14]), differentially expressed genes between cases and controls was calculated using DESeq2. Within each disease and location, genes were considered validated if they: 1. Were significantly different between disease and control in the main cohort, with FC ≥1.5 and FDR ≤0.05. 2. Changed in the same direction in both main and validation cohorts. 3. Were significantly different between disease and control in the validation cohort, with FC ≥1.2 and FDR ≤0.1. Identifying similar genes between diseases and locations: A list of all genes that passed validation in at least one disease was compiled. Within this atlas, a gene is considered similar between diseases and locations if it: 1. Changes in the same direction in all main cohorts. 2. Is significantly different between disease and control in all main cohorts, with FC ≥1.2 and FDR ≤0.1. This analysis was performed separately for lncRNA and protein-coding genes.

Microbiome 16S rRNA Analysis and Association with Genes

16S rRNA reads were processed in a data curation pipeline implemented in QIIME 2 version 2021.4. Reads were demultiplexed according to sample specific barcodes. Quality control was performed by truncating reads after three consecutive Phred scores lower than 20. Reads with ambiguous base calls or shorter than 150 bp after quality truncation were discarded. Amplicon sequence variants (ASVs) detection was performed using Deblur, resulting in 156 samples with median of 28,504 reads/sample (IQR 18,980-43,470). ASV taxonomic classification was assigned using a naive Bayes fitted classifier, trained on the August 2013 Greengenes database for 99% identity 150 bp long reads. Severity associated ASVs: differentially abundant ASVs (between mild and severe UC patients) were identified using a paired feature-wise non-parametric rank mean test as implemented in Calour with dsFDR multiple hypothesis correction (FDR <0.1). For each feature (bacteria), the relative abundance across all samples is ranked. The p-value is calculated by comparing the mean of the ranks for the bacteria in each group to random permutations of the group labels, that are performed only within samples of similar pairing field values. Finally, dsFDR multiple hypothesis correction is applied for the p-values resulting from all the features. Hitmap was generated using Calour with default parameters. Identifying association between lncRNA and microbial ASV: “Hierarchical All-against-All significance testing” (HAllA, http://huttenhower.sph.harvard.edu/halla) version 0.8.20 was used to identify potential correlations between lncRNAs and ASVs associated with disease severity, using Spearman's correlation. 59 mild and severe UC associated ASVs, and the top 30 mild and severe UC associated lncRNAs, prioritized by DEseq2 FDR were used as input for the HAllA pipeline, with an FDR of 0.1.

Cell Culture and Organoids

Caco2 and HT29 human colon carcinoma cell lines were purchased from the American Type Culture Collection (Manassas, VA, USA) and maintained in standard culture conditions in DMEM (GIBCO 41965-039, Scotland) containing 10% (for HT-29) and 20% (for Caco2) (v/v) heat-inactivated fetal bovine serum (GIBCO 12657-029, Scotland). Cells were maintained at 37° C. in a humidified atmosphere containing 5% CO2. Hypoxic conditions were obtained by H₂O₂ [200 μM] or DFO [100 μM] and inflammation conditions by LPS [100 ng/ml]+IFNγ [40 ng/ml] for 24 hr.

Crypts isolation and organoids culturing: L-WRN medium (L-WRN media) was generated using the ATCC mouse fibroblasts cells (L-WRN cells-CRL-3276™), that produce Wnt-3A, R-spondin 3, and noggin. To maintain proliferation, TGFBIR inhibitor (SB431542) is added. To initiate differentiation, EP4 inhibitor (L-161,982) is added to DMEM-F12 (Gibco 12634010) medium without FBS. Intestinal biopsies from patients undergoing evaluation via colonoscopy according to approval by the Ethics Committees of the Sheba Medical center were used, after written informed consent were obtained from patient and/or families Two biopsies were used for crypts isolation. Biopsies were washed 3 times with PBS and Gentamicin/Amphotericin (X 500—corning), cut to small pieces and incubated in cold for 30 minutes with Gentle Cell dissociation Reagent (STEM CELL 100-0485) that helps to release the crypts. Isolated crypts were seeded in Matrigel (Corning 354234) and L-WRN medium to generate the organoids. Cells were maintained at 37° C. in a humidified atmosphere containing 5% CO2 and were passaged weekly. IFNγ (25 ng/ml), and TNFα (20 ng/ml) were applied after differentiation for 48 h for an additional 24 h in the differentiation conditions.

Knockdown by Short Hairpin RNA (shRNA):

The shRNAs were designed by rnaidesigner tool: https://rnaidesigner.thermofisher.com and cloned into pLKO.1 (#8453) plasmid. The shRNA plasmids were packaged into virions and generated in 293T cells. Then, Caco2 cells were infected by lentiviruses with selection using puromycin. shRNA sequences can be found in Table 1.

TABLE 1
siRNA/shRNA sequences

| | Sense | Antisense |
|---|---|---|
| GATA6- | GCGCATTCGATCGCATTCAGG | CCTGAATGCGATCGAATGCGC (SEQ ID |
| AS1 sh1 | (SEQ ID NO. 16) | NO. 17) |
| GATA6- | GGAGTCGCGCATTCGATCGCA | TGCGATCGAATGCGCGACTCC (SEQ ID |
| AS1 sh2 | (SEQ ID NO. 18) | NO. 19) |
| TGM2 si1 | CAGTTTGAAGATGGGATCCTAGACA | TGTCTAGGATCCCATCTTCAAACTG (SEQ |
| | (SEQ ID NO. 20) | ID NO. 21) |

Transfection with siRNA: GATA6-AS1 shRNA stable cells (3.5×10⁵/6 well, 8×10³/96 well) were seeded in plates and allowed to adhere for 24 hr. The next day, cell monolayers were transfected with 30 pmol siRNA specific for TGM2, or control siRNA, using RNAiMAX lipofectamine (Thermpfisher, 13778075) according to manufacturer's protocol. The cells were incubated for 24 hr. after transfection and were prepared for further analysis.

JC1 Mitochondrial Membrane Potential Determination Caco2 Cells

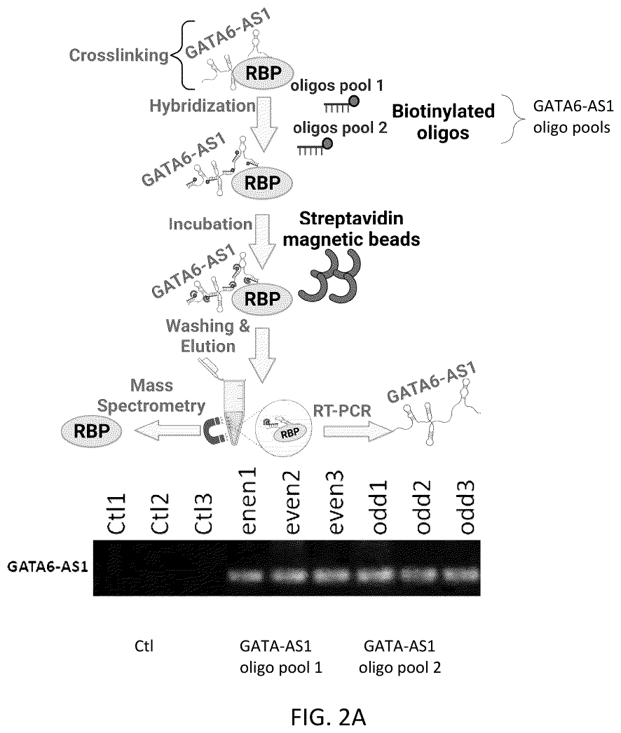

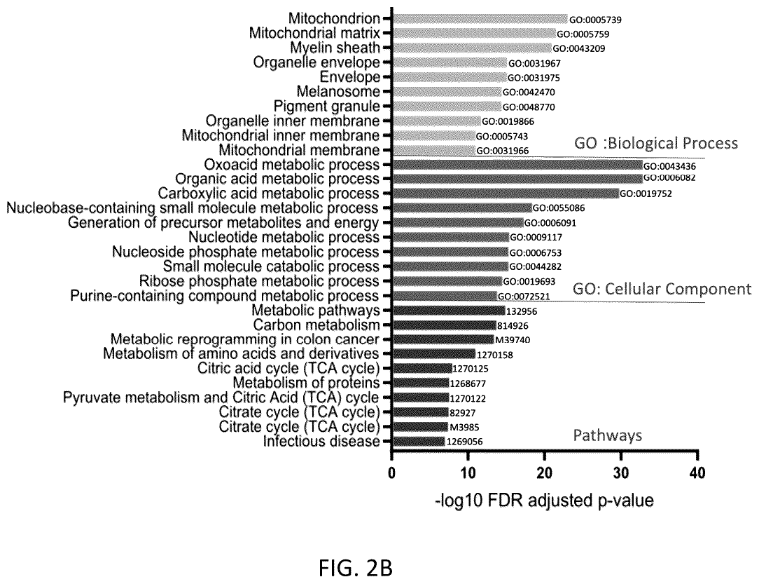

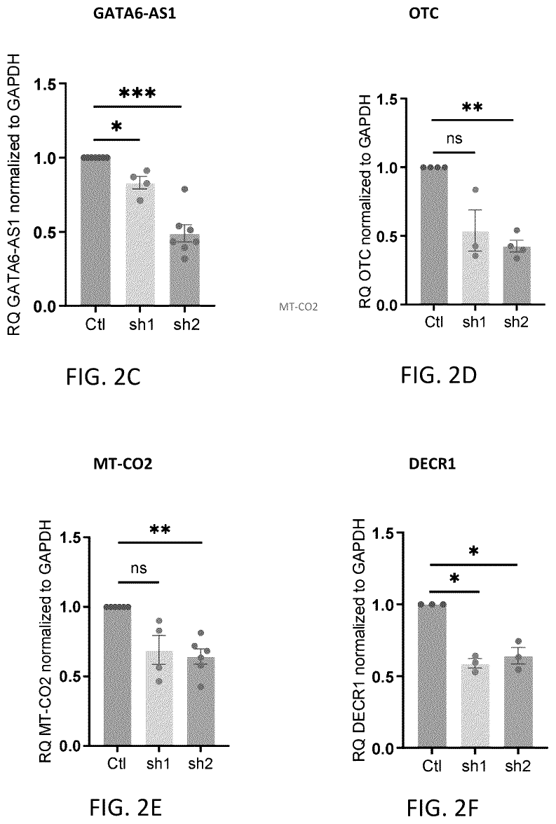

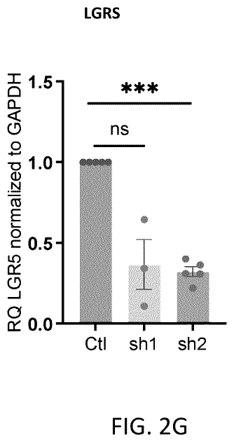

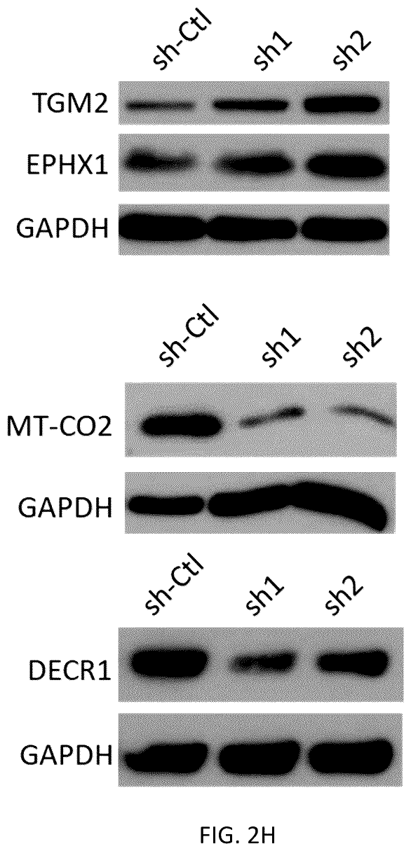

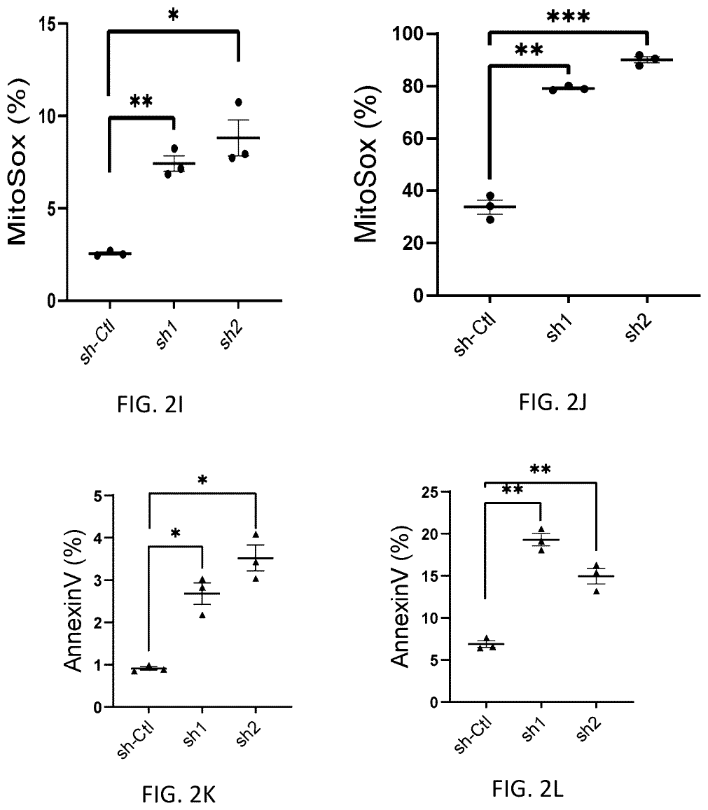

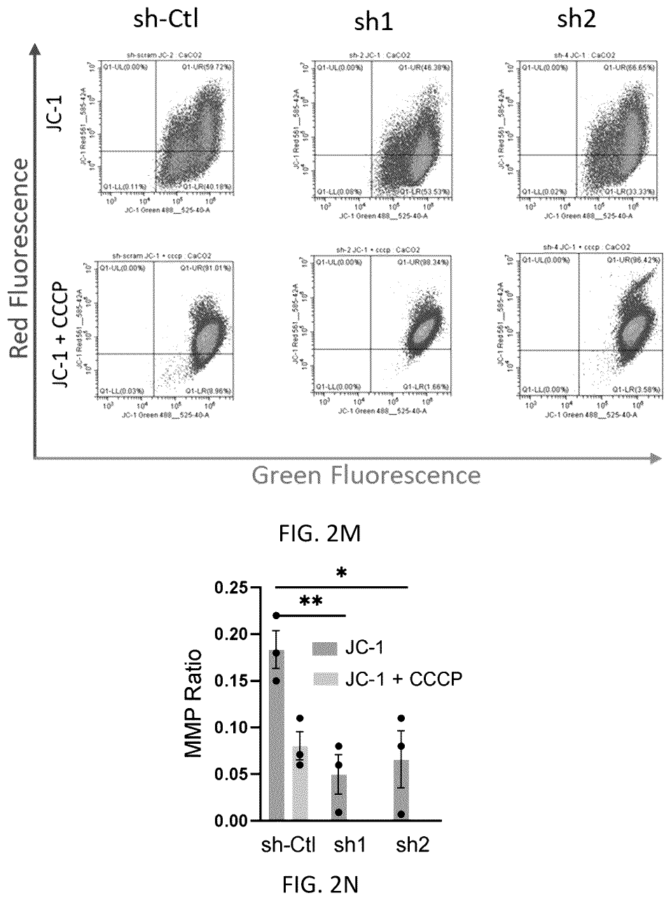

JC1 staining was performed on GATA6-AS1 knock down and control Caco-2 cells using the JC-1 (5,5″,6,6″-tetrachloro-1,1″,3,3″-tetraethylbenzimidazolylcarbocyanine iodide, MITOPROBE JC-1 ASSAY KIT, M34152, Rhenium, Inc. Eugene, OR) reagent according to the manufacturer's instructions. In brief, JC-1 dye was added at 2 μM to washed cells, and incubated for 15 minutes at 37° C., 5% CO2. Cells were washed, acquired on a CytoFLEX flow cytometer, and data were analyzed using CytExpert software. The MMP was calculated as the ratio of red-MFI/green-MFI in Caco2 cells. Representative data is shown in FIG. 2M-N. As a positive control for the specificity of the assay 50 mM of CCCP (carbonyl cyanide 3-chlorophenylhydrazone) were used to depolarize the mitochondrial membrane potential measured using the JC-1 dye, and incubated for 5 min at 37° C., 5% CO2.

RNA Isolation, Quantitative PCR (Real-Time RT-PCR)

Total RNA was isolated using Tri Reagent-LS (Sigma, T9424, Saint Louis, MO, USA). RNA concentration and purity were assessed on a NanoDrop ND-1000 spectrophotometer (Thermo Fisher Scientific, Wilmington, DE, USA). First-strand cDNA was synthesized using a high-capacity RNA-to-cDNA reverse transcription kit (Applied Biosystems, 4387406). Quantitative real time polymerase chain reaction (qRT-PCR) was performed using a Fast SYBR Green Master mix (Applied Biosystems, 4385612) and qRT-PCR machine with standard qRT-PCR parameters to analyze the expression of indicated genes compared with the control gene GAPDH. Results were analyzed with the comparative CT method, and log 10 (relative quantification values [Rq] values) are shown. All qRT-PCR reactions were performed in triplicates and the statistics were performed on dCt Mean values. The primers are listed in

TABLE 2
qPCR primers

| | | | Product |
|---|---|---|---|
| Gene | For (5′-3′) | Rev (5′-3′) | size |
| GATA6- | TTCTGGGAGTCGCGCATT (SEQ ID NO. | GTGGCCGCATTTGGAAAA (SEQ ID NO. | 121 |
| AS1 | 22) | 23) | |
| GATA6 | TTCCCATGACTCCAACTTCC (SEQ ID NO. | CGCCTATGTAGAGCCCATCT (SEQ ID | 180 |
| | 24) | NO. 25) | |
| OTC | GCTGGCTAACTTGCTGTGG (SEQ ID NO. | GCCCTTCAGCTGCACTTT (SEQ ID NO. | 180 |
| | 26) | 27) | |
| MT- | CTGAACCTACGAGTACACCG (SEQ ID | TTAATTCTAGGACGATGGGC (SEQ ID | 326 |
| CO2 | NO. 28) | NO. 29) | |
| DECR1 | GTGTGATGTGAGGGATCCTG (SEQ ID | TAGTGTCACGAAGGCTGTGC (SEQ ID | 187 |
| | NO. 30) | NO. 31) | |
| IL8 | GGAGAAGTTTTTGAAGAGGGCTGAGAA | CAGACCCACACAATACATGAAGTGTT | 129 |
| | T (SEQ ID NO. 32) | G (SEQ ID NO. 33) | |
| LGR5 | GGTGACAACAGCAGTATGGACGAC | CAGCCAGCCATCAAGCAGGT (SEQ ID | 186 |
| | (SEQ ID NO. 34) | NO. 35) | |
| GAPDH | TGGACCTCATGGCCCACA (SEQ ID NO. | TCAAGGGGTCTACATGGCAA (SEQ ID | 169 |
| | 36) | NO. 37) | |
| TGM2 | GGCATGGTCAACTGCAAC (SEQ ID NO. | CAGCACTGGCCATACTTGAC (SEQ ID | 158 |
| | 38) | NO. 39) | |

lncRNA Pull Down and Mass Spectrometry

Tilling anti-sense oligos with BiotinTEG at the 3-prime end were designed using https://www.biosearchtech.com/chirp-designer. Oligos were then separated into two pools of “even” and “odd” oligos pools, which specifically target GATA6-AS1. The experiments were performed using both pools, which serve as internal controls for each other, and real RNA-dependent signals would be present from both pools. The two pools of oligo probes were incubated with cross-linked and sheared lysates of differentiated Caco2 cells. Hybridized oligos were subsequently subjected to streptavidin agarose beads. Beads are washed and RNA and protein were recovered. Proteins were trypsin-digested on the streptavidin beads, followed by their purification on C18 StageTips (Pierce c18 spin tips 84850). The retrieved peptides (GATA6-AS1 lncRNA pull-down) were analyzed by mass-spectrometry at the Proteomic unit at the Sheba medical center. Purified peptides were separated on EasySpray columns (50 cm long 0.75 μm 803A PepMap) using a 140 min water-acetonitrile gradient and were injected to the Q-Exactive HF mass spectrometer (Thermo Scientific) via the EasySpray ionization source. MS analysis was performed using data-dependent acquisition, with fragmentation of the top 10 proteins from each MS spectrum. MS spectra were acquired with a 60,000 resolution and MS/MS spectra were acquired with a 15,000 resolution. MS raw files were analyzed with MaxQuant version (1.5.8.0) and the Andromeda search engine, using the Uniprot database (UP000005640 2016). Search engine included oxidation and N-term acetyl as variable modifications and carbamidomethyl as fixed modifications and used FDR of 0.01 for PSM and protein identification. The label-free quantification (LFQ) algorithm in MaxQuant was used for relative quantification of proteins. MaxQuant identified a total of 2317 proteins, and these were filtered to remove reverse and contaminant proteins, and those that were identified only by their modification site. 1743 of 2317 proteins were additionally filtered based on valid values to retain only proteins present in at least ⅓ of all samples (valid log 2 intensity). The missing values were then inputted only in the control using constant value (lower value identified) and the FC was calculated by subtracting the control average values from GATA6-AS1 associated protein results. One sample t-test looking at higher binding to GATA6-AS1 oligos vs. controls with delta label-free quantitation (LFQ) intensity ≥0.1 and FDR ≤0.05 was performed and identified 285 proteins. Oligos are listed in Table 3.

TABLE 3
GATA6-AS1 RNA pull-down oligos

| | Even (5′-3′) | Odd (5′-3′) |
|---|---|---|
| 1 | CCTCGTTAGGTTGGAAAACC (SEQ ID NO. 40) | CTCGTACAGTTGATTCAGAC (SEQ ID NO. 41) |
| 2 | TATCGAGTGTCCAGGGATAT (SEQ ID NO. 42) | ATCTGGAGGGTAAAATGCCG (SEQ ID NO. 43) |
| 3 | ATTCCAGAGTTTTCTACCTT (SEQ ID NO. 44) | AAAAGCAAGGCATTCCGAGT (SEQ ID NO. 45) |
| 4 | CACCATCTCTGGAAACCATA (SEQ ID NO. 46) | ACCGTTCTCATCCAATTTAG (SEQ ID NO. 47) |
| 5 | AGAAACTCTCCAGGGACATC (SEQ ID NO. 48) | AGGACAAGAGGCGTTGTTTT (SEQ ID NO. 49) |
| 6 | GGCAAAAAGCGCATTTGCTG (SEQ ID NO. 50) | CAAAGGTCAGCTGGGGAATG (SEQ ID NO. 51) |

Immunoblotting

For western blot analysis, cells were lysed on ice, in RIPA lysis buffer [R0278-50ML, Sigma] in the presence of protease inhibitor cocktail (Roche). Equal amounts of protein, as determined by the Bradford assay (B6916, Sigma), were resolved by electrophoresis in SDS 10% polyacrylamide gel and then transferred to a cellulose nitrate membrane (10401383, Tamar). The membrane was incubated with one of the following primary antibodies: anti-TGM2 (ab2386, Abcam); anti-EPHX1 (sc-135984, Santa-Cruz); anti-DECR1 (HPA023238, Sigma), anti-GATA6 (HPA066629); anti-HIF1a (14179S, Cell signaling); anti-GAPDH (#2118 Cell signaling). Binding of the primary antibody was detected using an enhanced chemiluminescence kit (XLS142,0250, Cyanagen).

RNA Fluorescent In Situ Hybridization (FISH)

Caco2 cells were seeded on 18 mm glass coverslips in a 12-well plate. The cells were fixed with 1 ml of Stellaris fixation buffer (3.7% Formaldehyde, PBS, Nuclease-free water) for 10 min, washed twice with PBS, then stored in 70% ethanol at 4° C. The ethanol was removed, and 1 ml wash buffer A (20% formamide in Stellaris RNA FISH Wash Buffer A (Biosearch Technologies)) was added. After incubation for 5 min at RT, a 100 ul drop of hybridization buffer (20% formamide in Stellaris RNA FISH Hybridization Buffer (Biosearch Technologies)) containing the probe (375 nM) was added onto a parafilm. The coverslip was transferred to the drop with cells facing down and incubated in the dark up to 16 hours at 37° C. in a humidified chamber. On the next day, the coverslips were transferred to a new 12-well plate containing 1 ml wash buffer A and incubated in the dark at 37° C. for 30 min. For staining the nuclei, the wash buffer was aspirated and 1 ml Hoecht solution (wash Buffer A consisting of 5 ng/ml Hoecht) was pipetted in each well and incubated for additional 30 min in the dark at 37° C. After removing the staining solution, 1 ml wash buffer B (Biosearch Technologies) was added and incubated for 5 min at RT. Confocal imaging was performed with a Carl Zeiss confocal microscope using a 63× oil immersion objective.

Cell Bioenergy Tests

Mito Stress Test and Glycolysis Stress Test were done using the Seahorse XFe96 Analyzer (Agilent Technologies). GATA6-AS1 shRNA/sh-Ct1 stable Caco2 cells were seeded in XFe 96-well microplates (8,000 cells/well) (Agilent Technologies) with/without DFO (100 μM)+LPS (100 ng/ml)+IFNγ (40 ng/ml) treatment (for 24 hr), or via TGM2 knockdown by siRNA the day prior to the experiment. The XFe96 cartridge was equilibrated with the calibration solution (Agilent technologies) overnight at 37° C. At the day of the experiment, cells were washed and incubated in unbuffered assay media (Sigma, cat #D5030), pH 7.4, supplemented with 5 mM glucose (Sigma, cat #G8270), 1 mM pyruvate (Sigma, cat #P8754) and 2 mM Glutamine (Sigma, cat #G3126) for Mito stress test, or with glutamine only for glycolysis stress test, for 1 hour at 37° C. non-CO2 incubator. For both assays, the compounds were prepared from DMSO stocks, using the assay media for dilution. For Mito stress test, the following compounds and final concentrations were used: 1 μM oligomycin (Sigma cat #75351), 0.5 μM FCCP (Sigma cat #C2920), 2 μM antimycin A and 2 μM rotenone (Sigma, cat #R8875 and A8674 respectively). For Glycolysis stress test: 10 mM glucose, 1 μM oligomycin and 50 mM 2-DG (Sigma, cat #D3179) were used. Measurement parameters were 3 minutes mixing time and 3 minutes measuring time. Protein quantification for each well was done at the end of the assay using Bradford assay kit (B6916, Sigma). Data were normalized to μg protein.

Extraction of Cell Lines for Metabolomic Analysis

Cells (5×100 were plated onto 6-well plates and cultured in standard medium for 24 h, the next day hypoxia and inflammation conditions were induced (as described above). For intracellular metabolomic analysis, cells were quickly washed two times with cold PBS (4° C.). PBS was thoroughly aspirated and precooled (−20° C.) Extraction solution (ES: 50% methanol, 30% acetonitrile, 20% water) was added to the plates in a ratio of 1 ml of ES per 1×10⁶ cells and placed on a rocking shaker at 4° C. for 10 min. The extraction solution from each well was collected into a microcentrifuge tube, vortexed, centrifuged at 14,000 g for 15 min at 4° C. and stored at −80° C. until submission for LC-MS metabolomics analysis. For extracellular analysis: media from each well was collected and extracted with ES in a ratio of 1:50 (media:ES). The samples' protein quantity was measured by Bradford assay for normalization purposes.

LC-MS Metabolomic Analysis

LC-MS analysis was conducted as described (Mackay et al., Methods Enzymol 561, 171-196, (2015)). Briefly, Dionex Ultimate ultra-high-performance liquid chromatography (UPLC) system coupled to Orbitrap Q-Exactive Mass Spectrometer (Thermo Fisher Scientific) was used. Resolution was set to 35,000 at 200 mass/charge ratio (m/z) with electrospray ionization and polarity switching mode to enable both positive and negative ions across a mass range of 67-1000 m/z. UPLC setup consisted of a ZIC-pHILIC column (SeQuant; 150 mm×2.1 mm, 5 μm; Merck). 5 μl of cells extracts were injected and the compounds were separated using a mobile phase gradient of 15 min, starting at 20% aqueous (20 mM ammonium carbonate adjusted to pH 9.2 with 0.1% of 25% ammonium hydroxide):80% organic (acetonitrile) and terminated with 20% acetonitrile. Flow rate and column temperature were maintained at 0.2 ml/min and 45° C., respectively, for a total run time of 27 min. All metabolites were detected using mass accuracy below 5 ppm. Thermo Xcalibur 4.1 was used for data acquisition. The peak areas of different metabolites were determined using Thermo TraceFinder™ 4.1 software, where metabolites were identified by the exact mass of the singly charged ion and by known retention time, using an in-house MS library built by running commercial standards for all detected metabolites. Each identified metabolite intensity was normalized to μg protein. Metabolite-Auto Plotter (Pietzke and Vazquez, Cancer Metab 8, 15, (2020) was used for data visualization during data processing. Normalized metabolites (each metabolite value was divided by the sum of total metabolites value) framework was used for differential expression analyses. Differential expression analysis was based on a paired analyses using rank mean test and FDR<0.1.

ROS and Apoptosis

Mitochondrial ROS levels were assessed using MitoSOX Red (M36008, ThermoFisher) assay, a redox-sensitive fluorescent probe that is selectively targeted to the mitochondria, in combination with Annexin V-CF647 (diluted 1:50). Cells were incubated with 5 μM MitoSOX Red probe for 20 minutes at 37° C. The cells were washed twice with PBS, and acquired on Navios flow cytometer, the data were analyzed using Kaluza Software.

Statistical Analysis

Overall, Spearman's rank correlation was used for continuous variables and Mann-Whitney U test for categorical variables, with Benjamini-Hochberg Procedure for FUR correction. For the tissue culture cellular data, 2-sided T test P<0.05, **P<0.01, ***P<0.001, was used and statistical analyses were performed in GraphPad Prism v9.31.

### Example 1: GATA6-AS1 is Dysregulated in Celiac Duodenum, Crohn's Disease Ileum, and UC Rectum

A comprehensive atlas of lncRNAs and protein-coding genes that are dysregulated in celiac duodenum, CD ileum, and UC rectum was generated using a unified pipeline including altogether 673 samples of mRNA mucosal biopsies in both main and validation cohorts (as described in methods, above). lncRNAs and protein-coding genes were considered validated if they showed similar direction of change and passed predefined fold change and FDR in test and validation cohorts (as described in methods, above). Under these criteria, 37 lncRNAs and 817 protein-coding genes that showed similar dysregulation in the three diseases; celiac disease, CD, and UC were identified.

Within the 37 lncRNAs dysregulated in the 3 disease, GATA6-AS1 exhibited the highest M1 MM score (indicative of centrality in M1 module, MM=0.84), a module that was also linked with all 3 UC outcome measurements in PROTECT.

### Example 2: GATA6-AS1 is Expressed in Epithelia, Linked with Epithelial Metabolic Functions, and with UC Outcome

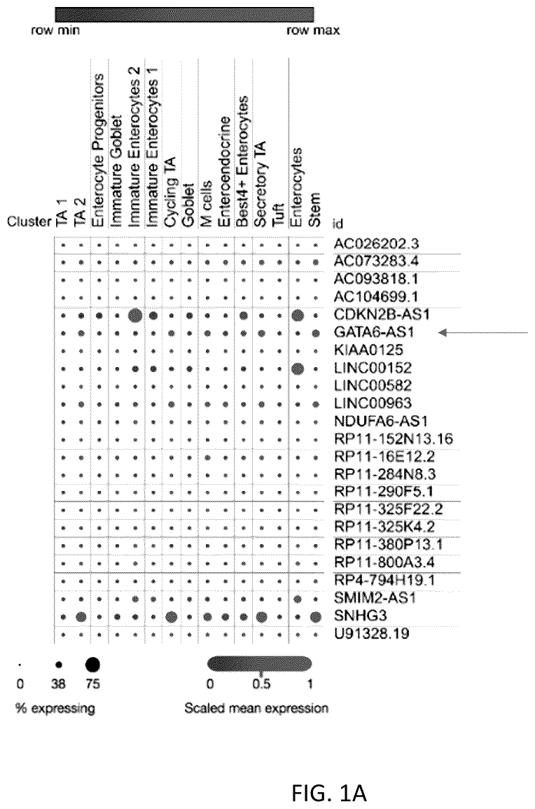

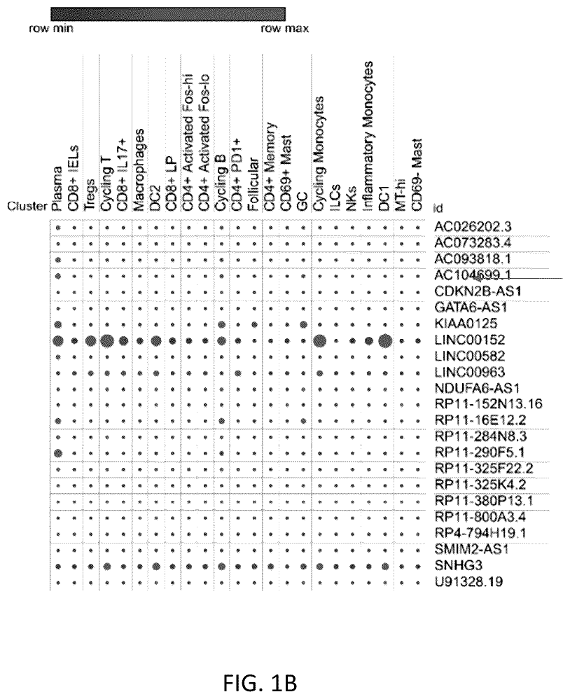

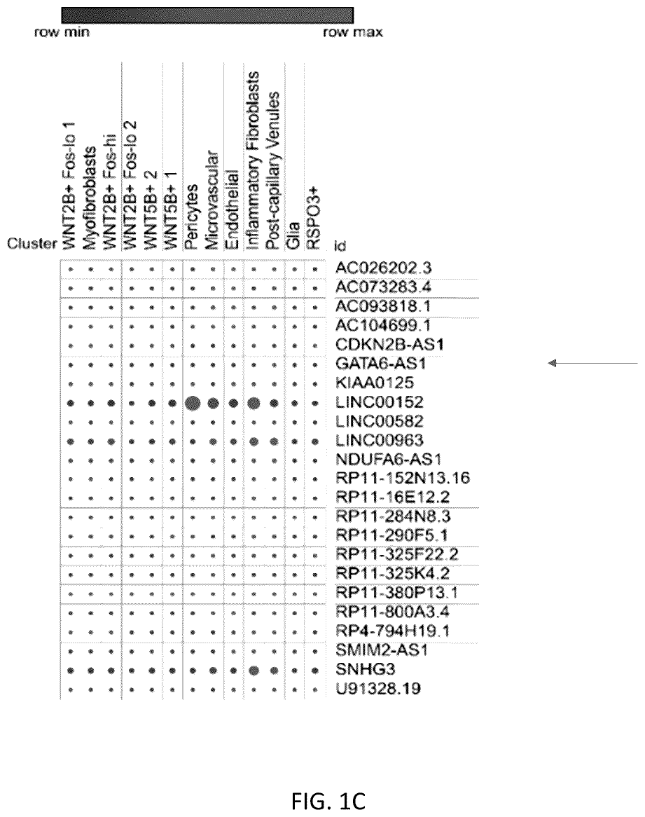

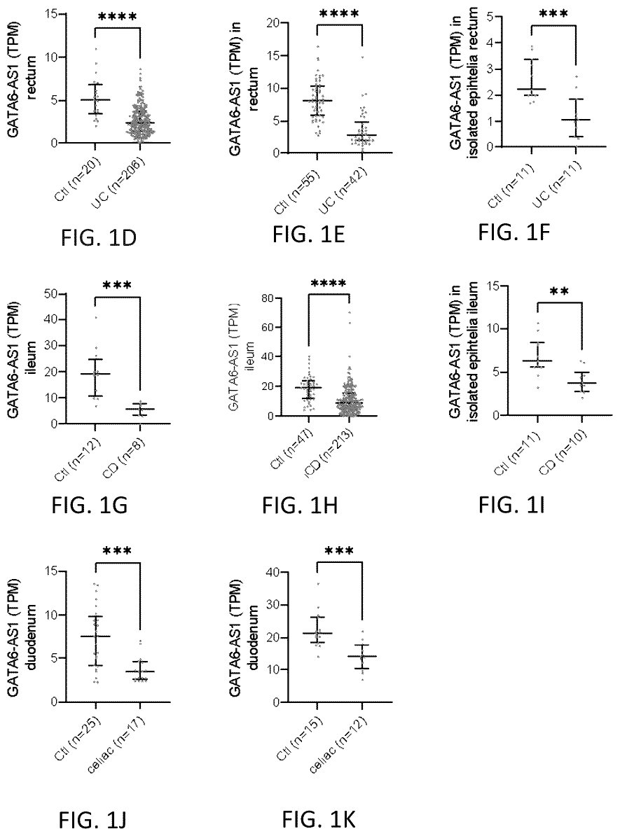

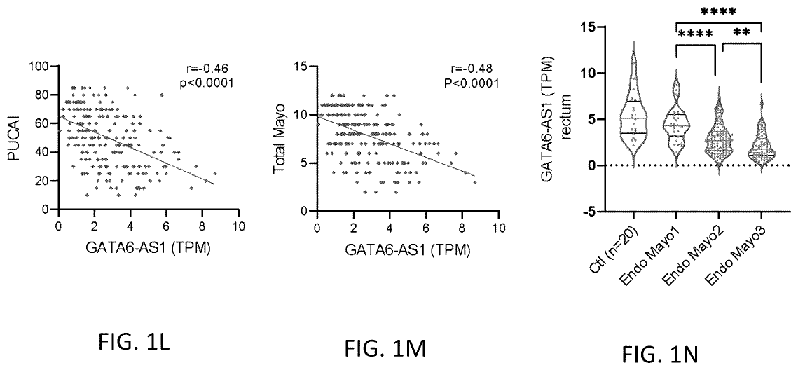

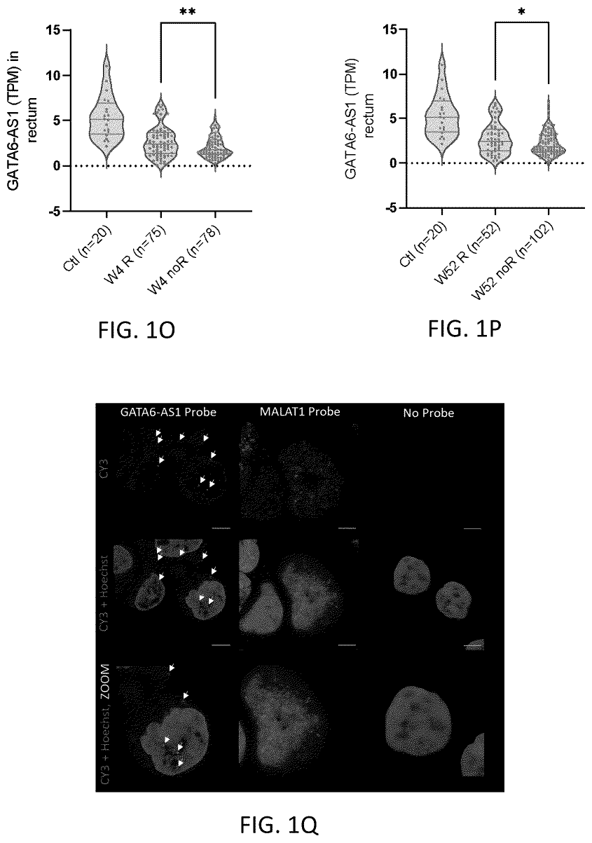

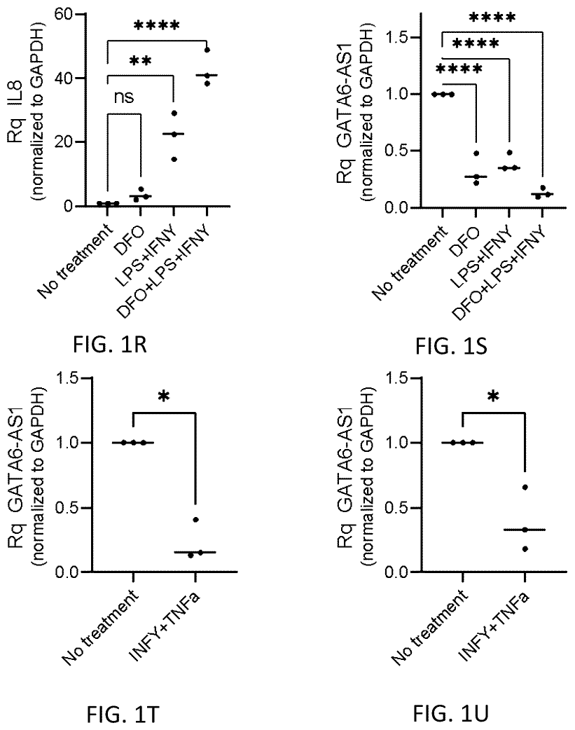

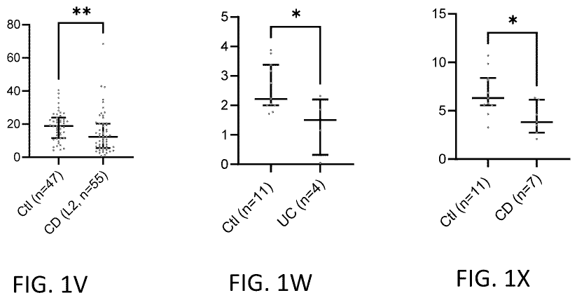

To examine cell-specific trends in gene expression, a publicly available adult human colon UC single-cell dataset was analyzed. This analysis confirmed expression of several lncRNAs including GATA6-AS1 to gut epithelia (FIG. 1A-C). To direct the mechanistic epithelial studies of GATA6-AS1, functional annotation enrichment of central blue module hub genes [top 10% with highest module membership (MM) score] that included GATA6-AS1 was used, and significant enrichments for cellular respiration, tricarboxylic acid cycle (TCA or Krebs cycle), mitochondrial membrane, acyltransferase activity, and lipid metabolism were noted. Reduction of GATA6-AS1 in UC (FIG. 1D-F), CD (FIG. 1G-I), and celiac (FIG. 1J-K) bulk biopsies (test and validation cohorts) and in a third cohort that used isolated epithelia from UC and CD (FIGS. 1F and 1I, respectively) was confirmed. GATA6-AS1 was negatively associated with both clinical and endoscopic UC severity (PUCAI (FIG. 1L), total Mayo (FIG. 1M), and endoscopic Mayo scores (FIG. 1N)), and with poorer outcome (noW4 and noW52SFR, FIGS. 1O and 1P, respectively). Unlike MALAT1 that had predominant nuclear expression, GATA6-AS1 is present in the cytoplasm and the nucleus as indicated by FISH (FIG. 1Q) and fractionation assays. Providing inflammatory triggers, namely LPS, IFNγ and deferoxamine (DFO), known to upregulate hypoxia-inducible factor-1 alpha that is also upregulated in IBD [9, 15], resulted in GATA6-AS1 reduction in epithelia tissue culture model (FIG. 1R-S). Similar reduction was observed in primary ileum and rectal organoids from control cases after triggering with TNFα+IFNγ (FIG. 1T-U). GATA6-AS1 was also reduced in non-inflamed ileum of CD and in isolated epithelia cells from CD ileum and UC rectum of non-inflamed samples (FIG. 1V-X).

### Example 3: GATA6-AS1 Regulates Epithelial Mitochondrial and Metabolic Functions

Mass spectrometry analysis of the GATA6-AS1 protein interactome was performed using affinity capture of the endogenous GATA6-AS1 RNA by two independent tiling GATA6-AS1 antisense-oligos pools. This analysis identified 285 proteins (FDR<0.05). Functional annotation of these proteins indicated strong enrichment of epithelial metabolism, mitochondria, and TCA cycle genes and functions (FIG. 2A-B). Such proteins included EPHX1, a microsomal epoxide hydrolase, and DECR1 a mitochondrial enzyme involved in unsaturated fatty acid oxidation, and TGM2 tissue transglutaminase, which is an autoantigen in celiac disease which was also induced in celiac, CD, and UC.

Based on the interactome data, it was functionally tested whether GATA6-AS1 regulates mitochondrial associated genes and functions. Stable silencing of GATA6-AS1 by up to 50% using two independent shRNA (sh1, sh2) resulted in reduction of nuclear-encoded mitochondria related OTC mRNA levels, and suppression of DECR1 which participates in the beta-oxidation in the mitochondria, and reduction in the mitochondrial encoded MT-CO2 (part of mitochondria complex IV) at the mRNA and protein levels (FIG. 2C-G). Reduced mRNA levels of LGR5, intestinal stem cell marker potentially linking mitochondrial genes and functions with epithelial renewal and in GATA6 level was also noted. In contrast, induction of EPHX1 and TGM2 at the protein level (FIG. 2H) was observed. As TGM2 overexpression increases ROS production [16], MitoSOX FACS assay was used to measure mito-ROS production, and AnnexinV to measure cell apoptosis in these cells. GATA6-AS1 silencing resulted in higher mitochondrial ROS production at basal conditions, which was further induced upon H₂O₂ treatment (FIG. 2I-J), coupled with increased cell apoptosis (FIG. 2K-L). MMP in control untreated cells was 0.22, and upon GATA6-AS1 suppression the MMP was reduced to 0.08, which is like the MMP with CCCP that is known to depolarize the mitochondrial membrane and reduce the MMP (FIG. 2M-N). MMP is calculated as the ratio of red/green fluorescence, whereby green fluorescence represents depolarized JC-1 monomer and red fluorescence represents polarized JC-1 polymer. The analysis is performed in the absence or presence of 50 μM CCCP (carbonyl cyanide 3 chlorophenylhydrazone) that reduces MMP (induces mitochondrial depolarization).

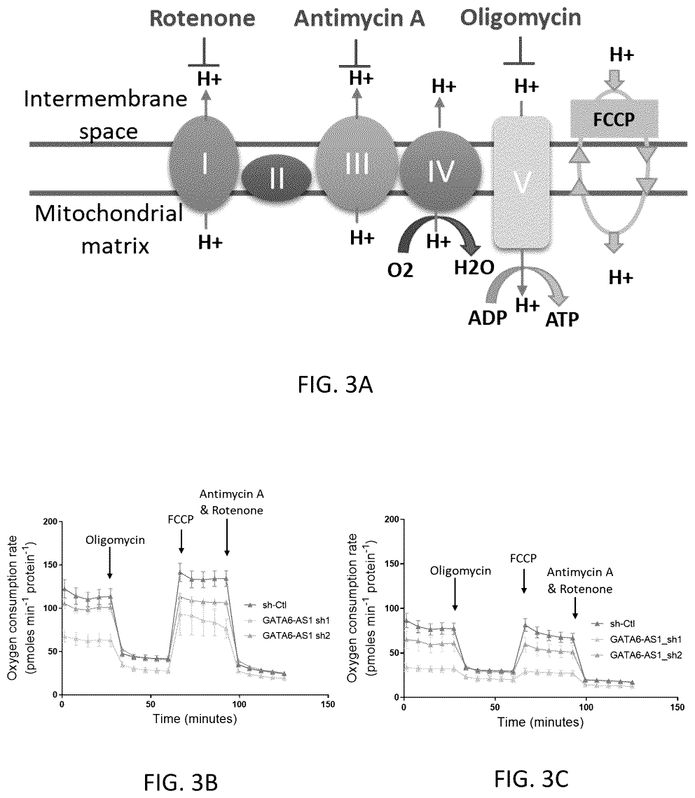

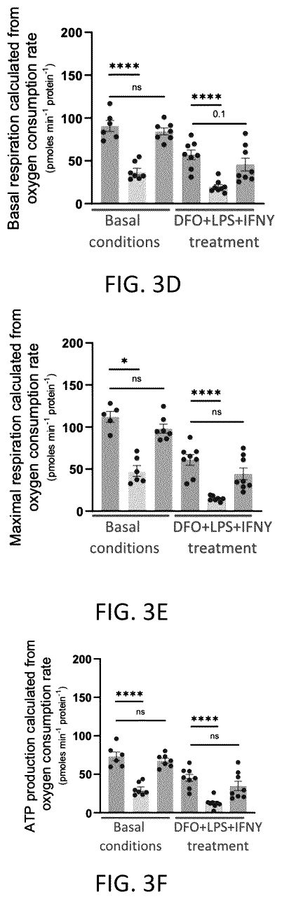

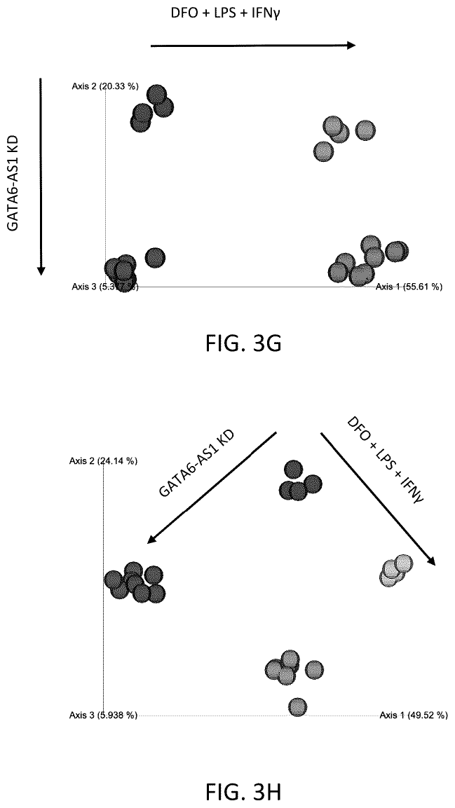

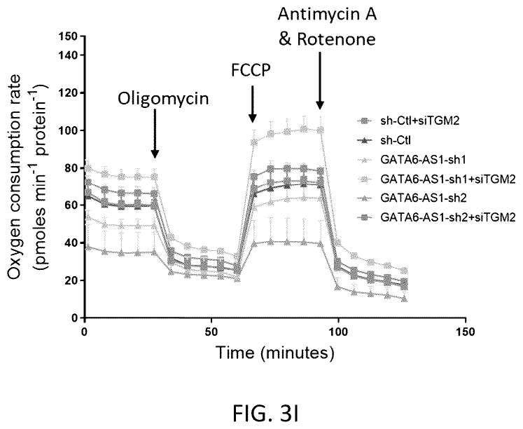

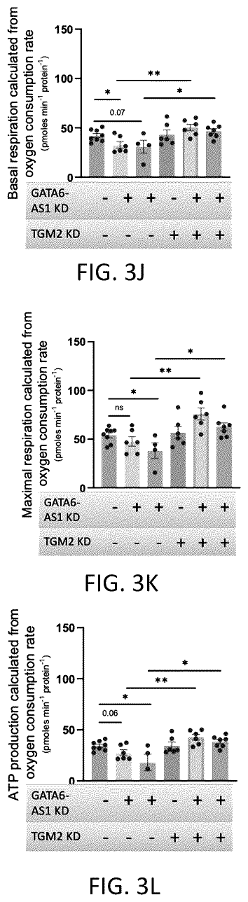

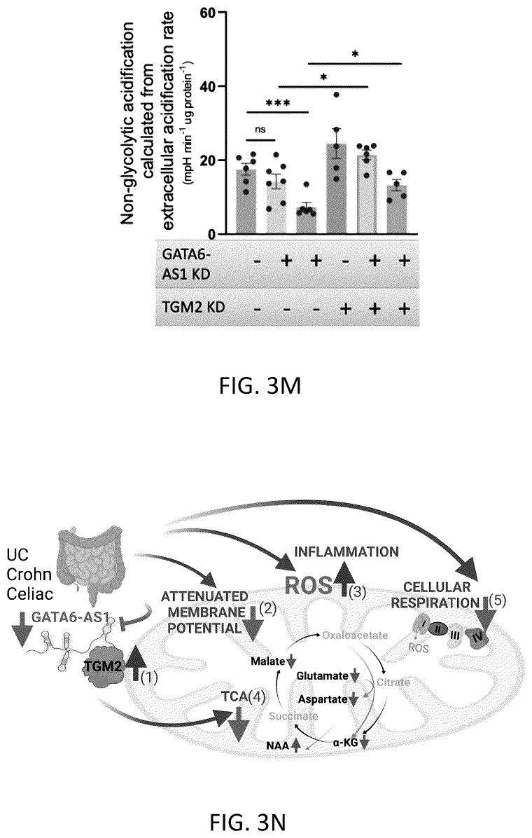

GATA6-AS1 silencing resulted in reduction in Oxygen Consumption Rate (OCR) measured by the Seahorse XFe with reduced basal and maximal respiration and ATP production, which were more pronounced in cells treated with DFO+LPS+IFNγ (FIG. 3A-F). To capture cells' metabolic state more globally, using a complementary approach, an untargeted metabolomics was applied on similar cells and their suspending media. This approach showed robust separation based on the metabolic profile in cells and in the media by treatment, but also by GATA6-AS1 knockdown (FIG. 3G-H), using unbiased unsupervised principal component analysis (PCA). 75 differentially expressed metabolites were identified in cells and 49 in their media (paired rank mean test, and FDR<0.1), with prominent reduction in TCA cycle metabolites α-ketoglutarate (aKG) and malate, and associated metabolites including glutamate, aspartate, N-acetyl-aspartate (NAA), and lactate upon GATA6-AS1 knockdown. This metabolomic data complements the results showing strong enrichment of GATA6-AS1 interactome with mitochondria metabolic function and TCA cycle, reduction of the mitochondrial membrane potential, and reduced cell respiration.

Next, it was examined whether reducing TGM2 in GATA6-AS1 knockdown cells could rescue the reduced cellular mitochondrial respiration (OCR) caused by GATA6-AS1 silencing. TGM2 co-silencing recovered the OCR levels of GATA6-AS1 knockdown cells to levels that were at least as high as in controls (FIG. 3I-L). Moreover, measuring extracellular acidification rate (ECAR) indicated reduction in non-glycolytic acidification linked with CO₂ from TCA cycle or breakdown of intracellular glycogen (i.e., glycogenolysis), and this was again rescued by reducing TGM2 in GATA6-AS1 knockdown (FIG. 3M). Taken together, these results indicate that GATA6-AS1 levels are reduced in gut epithelia of UC, CD, and celiac cases. Without wishing to be bound by theory, FIG. 3N is a schematic illustration showing that GATA6-AS1 is associated with a complex that includes TGM2 (1), that GATA6-AS1 silencing resulted in TGM2 induction (1), which resulted in attenuation of mitochondrial membrane potential (2), higher production of ROS (3), reduced cellular respiration (4), reduction of TCA metabolites (4), and reduced cellular respiration (5).

### Example 4: The Effect of GATA6-AS1 lncRNA-LNPs on UC Phenotypes in an Epithelial Tissue Culture Model

lncRNAs are added to the medium of tissue culture cells that stably express lower amounts of GATA6-AS1, as well as to the culture medium of primary stem-cell epithelial derived organoids, derived from human patients and healthy control subjects, both in 2D and 3D cultures. Different lncRNA amounts and concentrations are used, as well as different lipid nanoparticles. The interval needed to achieve durable lncRNA expression rescue and function is determined.

GATA6-AS1 lncRNAs at different titrating concentrations are packed in several LNP formulations using the NanoAssemblr microfluidic mixing system, in which mRNA molecules self-assemble with ionizable lipids under acidic conditions to form highly uniform nanoparticles. LNPs are then labeled for tracking and visualization by mixing 10%-20% Cy5-labelled RNA with non-labeled RNA, enabling tracking and visualization of intracellular encapsulation, while still maintaining nascent RNA functionality. The incorporation of lncRNA-LNPs in the epithelial models is assessed by determining lncRNA expression in cells. For example, 3D organoids are flipped “inside out” [17], wherein the lumen is exposed and lncRNA-LNPs internalization is measured through the apical epithelial compartment. Fractionation assays and direct fluorescence visualization (RNA-FISH) are used to specifically test if GATA6-AS1 lncRNA levels are increased in the cytoplasm or the nucleus. Similar methods are used for assessing RNA expression levels and localization following immune triggering with IFNγ and TNFα, for example.

Additionally, the effect of internalized GATA6-AS1 lncRNA-LNPs is assessed in cellular models, e.g., a GATA6-AS1 knockdown cell line, as described above. Reduction of GATA6-AS1 resulted in alterations in several mitochondrial metabolic functions (see FIGS. 2-3). Similarly, mitochondrial metabolic functions are assessed following addition of GATA6-AS1 lncRNA-LNPs to medium for evaluating rescue of aberrant phenotype. Toxicity is measured as well during cell proliferation, cell cycle, and apoptosis in epithelial models treated with GATA6-AS1 lncRNA-LNPs and those untreated. In case of low transfection efficacy, epithelial targeting using specific epithelial receptors (i.e., EpCAM) is used.

### REFERENCES

1 Ng, S. C. et al. Worldwide incidence and prevalence of inflammatory bowel disease in the 21st century: a systematic review of population-based studies. Lancet 390, 2769-2778, doi:10.1016/S0140-6736(17)32448-0 (2017).
2 Singh, P. et al. Global Prevalence of Celiac Disease: Systematic Review and Meta-analysis. Clin Gastroenterol Hepatol 16, 823-836 e822, doi:10.1016/j.cgh.2017.06.037 (2018).
3 Ardizzone, S. et al. Mucosal Healing Predicts Late Outcomes After the First Course of Corticosteroids for Newly Diagnosed Ulcerative Colitis. Clin Gastroenterol H 9, 483-U117, doi:10.1016/j.cgh.2010.12.028 (2011).
4 Rutgeerts, P. et al. Infliximab for induction and maintenance therapy for ulcerative colitis. N Engl J Med 353, 2462-2476, doi:10.1056/NEJMoa050516 (2005).
5 Neurath, M. F. & Travis, S. P. Mucosal healing in inflammatory bowel diseases: a systematic review. Gut 61, 1619-1635, doi:10.1136/gutjn1-2012-302830 (2012).
6 Ulitsky, I. & Bartel, D. P. lincRNAs: genomics, evolution, and mechanisms. Cell 154, 26-46, doi:10.1016/j.cell.2013.06.020 (2013).
7 Wapinski, O. & Chang, H. Y. Long noncoding RNAs and human disease. Trends Cell Biol 21, 354-361, doi:50962-8924(11)00061-4 [pii] 10.1016/j.tcb.2011.04.001 (2011).
8 Haberman, Y. et al. Long ncRNA Landscape in the Ileum of Treatment—Naive Early—Onset Crohn Disease. Inflamm Bowel Dis 24, 346-360, doi:10.1093/ibd/izx013 (2018).
9 Haberman, Y. et al. Ulcerative colitis mucosal transcriptomes reveal mitochondriopathy and personalized mechanisms underlying disease severity and treatment response. Nat Commun 10, 38, doi:10.1038/s41467-018-07841-3 (2019).
10 Hyams, J. S. et al. Clinical and biological predictors of response to standardised paediatric colitis therapy (PROTECT): a multicentre inception cohort study. Lancet 393, 1708-1720, doi:10.1016/50140-6736(18)32592-3 (2019).
11 Hyams, J. S. et al. Clinical and Host Biological Factors Predict Colectomy Risk in Children Newly Diagnosed With Ulcerative Colitis. Inflamm Bowel Dis, doi:10.1093/ibd/izab061 (2021).
12 Smillie, C. S. et al. Intra- and Inter-cellular Rewiring of the Human Colon during Ulcerative Colitis. Cell 178, 714-730 e722, doi:10.1016/j.ce11.2019.06.029 (2019).
13 Howell, K. J. et al. DNA Methylation and Transcription Patterns in Intestinal Epithelial Cells From Pediatric Patients With Inflammatory Bowel Diseases Differentiate Disease Subtypes and Associate With Outcome. Gastroenterology 154, 585-598, doi:10.1053/j.gastro.2017.10.007 (2018).
14 Leonard, M. M. et al. RNA sequencing of intestinal mucosa reveals novel pathways functionally linked to celiac disease pathogenesis. PLoS One 14, e0215132, doi:10.1371/journal.pone.0215132 (2019).
15 Haberman, Y. et al. Pediatric Crohn disease patients exhibit specific ileal transcriptome and microbiome signature. J Clin Invest 124, 3617-3633, doi:10.1172/JCI75436 (2014).
16 Ruan, Q., Quintanilla, R. A. & Johnson, G. V. Type 2 transglutaminase differentially modulates striatal cell death in the presence of wild type or mutant huntingtin. J Neurochem 102, 25-36, doi:10.1111/j.1471-4159.2007.04491.x (2007).
17 Haberman, Y. et al. Mucosal Genomics Implicate Lymphocyte Activation and Lipid Metabolism in Refractory Environmental Enteric Dysfunction. Gastroenterology 160, 2055-2071 e2050, doi:10.1053/j.gastro.2021.01.221 (2021).
18 Scanlon, S. A. & Murray, J. A. Update on celiac disease—etiology, differential diagnosis, drug targets, and management advances. Clin Exp Gastroenterol 4, 297-311, doi:10.2147/CEG.S8315 (2011).
19 Bray, N. L., Pimentel, H., Melsted, P. & Pachter, L. Near-optimal probabilistic RNA-seq quantification. Nat Biotechnol 34, 525-527, doi:10.1038/nbt.3519 (2016).
20 Soneson, C., Love, M. I. & Robinson, M. D. Differential analyses for RNA-seq: transcript-level estimates improve gene-level inferences. F1000Res 4, 1521, doi: 10.12688/f1000research.7563.2 (2015).
21 Co, J. Y. et al. Controlling Epithelial Polarity: A Human Enteroid Model for Host-Pathogen Interactions. Cell Rep 26, 2509-2520 e2504, doi:10.1016/j.celrep.2019.01.108 (2019).

1. A method of treating or preventing chronic intestinal inflammation and/or inflammatory bowel disease (IBD) in an individual in need thereof comprising administering GATA6-AS1 lncRNA or an expression vector encoding GATA6-AS1 lncRNA to said individual.

2. The method according to claim 1, wherein the individual has been diagnosed by a method that comprises measuring GATA6-AS1 lncRNA in the gastrointestinal tract of said individual, detecting a reduced level of GATA6-AS1 lncRNA as compared with a reference level, and thereby providing a positive diagnosis for chronic intestinal inflammation and/or IBD in the individual.

3. The method according to claim 1, wherein said GATA6-AS1 lncRNA comprises or consists of a sequence that is about 90-100% identical to any one of SEQ ID No. 1-15.

4. The method according to claim 1, wherein said GATA6-AS1 lncRNA is packaged in or attached to a delivery system.

5. The method according to claim 4 wherein said delivery system is selected from a group consisting of a viral-based delivery system, an exosome, a polymer-based delivery system, a lipid-based delivery system, and a conjugate-based delivery system.

6. The method according to claim 1, wherein said GATA6-AS1 lncRNA is administered by rectal administration, oral administration, or by injection.

7. The method according to claim 1, wherein the chronic intestinal inflammation or IBD is Crohn's disease (CD), ulcerative colitis (UC) or celiac.

8. The method according to claim 1, wherein the treatment is administered in combination with a TGM2 antagonist.

9. A method of increasing mitochondrial membrane potential and/or mitochondrial respiration in intestinal epithelial cells comprising administration of GATA6-AS1 lncRNA to an individual in need thereof.

10. A pharmaceutical composition comprising GATA6-AS1 lncRNA, or an expression vector encoding GATA6-AS1 lncRNA, and a pharmaceutically acceptable carrier.

11. The pharmaceutical composition according to claim 10 for treating or preventing chronic intestinal inflammation and/or inflammatory bowel disease (IBD) in an individual, and/or for increasing mitochondrial membrane potential and/or mitochondrial respiration in intestinal epithelial cells.

12. The pharmaceutical composition according to claim 11, wherein the individual has been diagnosed by a method that comprises measuring GATA6-AS1 lncRNA in the individual, detecting a reduced level of GATA6-AS1 lncRNA as compared with a reference level, and thereby providing a positive diagnosis for chronic intestinal inflammation and/or IBD in the individual.

13. The pharmaceutical composition according to claim 10, wherein said GATA6-AS1 lncRNA comprises or consists of a sequence that is about 90-100% identical to any one of SEQ ID No. 1-15.

14. The pharmaceutical composition according to claim 10, wherein said GATA6-AS1 lncRNA is packaged in or attached to a delivery system.

15. The pharmaceutical composition according to claim 14 wherein said delivery system is selected from a group consisting of a viral-based delivery system, an exosome, a polymer-based delivery system, a lipid-based delivery system, and a conjugate-based delivery system.

16. The pharmaceutical composition according to claim 10, wherein said GATA6-AS1 lncRNA, is administered by rectal administration, oral administration, or by injection.

17. The pharmaceutical composition according to claim 11, wherein the chronic intestinal inflammation or IBD is Crohn's disease (CD), ulcerative colitis (UC) or celiac.

18. The pharmaceutical composition according to claim 11, wherein the treatment is administered in combination with a TGM2 antagonist.

19. A kit comprising (i) means for determining the level of GATA6-AS1 lncRNA in an individual having or suspected of having or being at risk of developing chronic intestinal inflammation and/or IBD; and (ii) a pharmaceutical composition according to claim 10.
